(19) United States
(12) Patent Application Publication
(10) Pub. No.: US 2024/0366279 A1
(43) Pub. Date: Nov. 7, 2024

(54) NEUROMODULATION OF PHARYNGEAL NERVES TO TREAT DISEASES

(71) Applicant: Dorna HAKIMIMEHR, Redwood City, CA (US)

(72) Inventor: Dorna HAKIMIMEHR, Redwood City, CA (US)

(21) Appl. No.: 18/646,348

(22) Filed: Apr. 25, 2024

Related U.S. Application Data
(60) Provisional application No. 63/500,429, filed on May 5, 2023.
(60) Provisional application No. 63/591,569, filed on Oct. 19, 2023.

(51) Int. Cl. A61B 18/02; A61B 18/00

(52) U.S. Cl. CPC A61B 18/02; A61B 2018/00434; A61B 2018/00577

(57) ABSTRACT

Neuromodulation of nasal nerves to treat diseases is described where one example of a method for treating dysfunctions or diseases in a patient may generally include introducing a treatment device into a nasal cavity of a patient. The distal end of the treatment device may be navigated into proximity of nerve fibers passing through at least one of lesser palatine foramen, a palatovaginal foramen, or a sphenopalatine foramen, or anterior ethmoid foramen. The distal end may then be actuated to ablate all or substantially all of the nerve fibers such that the dysfunction or disease is mitigated.

### CROSS-REFERENCE TO RELATED APPLICATIONS

This application claims the benefits of priority to U.S. Prov. Apps. 63/500,429 filed May 5, 2023 and 63/591,569 filed Oct. 19, 2023, each of which is incorporated herein by reference in its entirety.

### FIELD OF THE INVENTION

The present invention is related to medical methods and apparatus. More particularly, the present invention is related to methods and devices to treat diseases by modulating the functioning of sensory neurons within the head and neck anatomy.

### BACKGROUND OF THE INVENTION

Sleep-disordered breathing is a common condition that encompasses a range of breathing disturbances during sleep, including obstructive sleep apnea (OSA) and non-obstructive sleep apnea. OSA is the most prevalent form of sleep-disordered breathing and is characterized by partial or complete obstruction of the upper airway during sleep. This obstruction leads to repetitive episodes of apnea, a complete cessation of airflow, or hypopnea, a partial reduction in airflow. OSA is associated with various comorbidities and consequences, including hypertension, cardiovascular disease, metabolic disorders, and cognitive impairment.

### Sensory Sensitization

Sensory neurons, also known as afferent neurons, are specialized nerves responsible for converting external and internal stimuli into electrical impulses that can be interpreted by the central nervous system (CNS). To interact with the environment, sensory neurons have specialized receptors to detect specific types of stimuli. Nociceptors can detect thermal, mechanical, or chemical changes that exceed a set threshold. Mechanoreceptors, register mechanical changes such as touch, pressure, vibration, and stretch, thermoreceptors are activated by changes in temperature, while chemoreceptors respond to changes in concentration of certain chemicals.

When a stimulus or tissue damage is sensed, receptors convert the encounter into an electrical signal which triggers an action potential. The potential propagates along the neuron's axon toward the CNS where it encounters a synapse; a junction between two neurons. Neurotransmitters released from the incoming neuron causes generation of an action potential in the postsynaptic neuron, propagating the signal deeper into the CNS. In response to the sensory input, CNS may elicit reactions by sending signals to muscles or glands. Upon activation, sensory neurons can release neuropeptides, such as substance P and calcitonin gene-related peptide (CGRP). These peptides promote vasodilation, leading to increased blood flow, which can aid in tissue repair and healing.

Neuropeptides released by sensory neurons can interact with immune cells, such as mast cells and macrophages which then release inflammatory mediators such as cytokines and chemokines. In turn, pro-inflammatory cytokines like IL-10, TNF-α, and IL-6 can affect neuronal excitability and synaptic transmission. This bidirectional communication between the nervous system and the immune system is crucial for maintaining homeostasis. Disruptions in neuroimmune crosstalk can contribute to various pathological conditions such as chronic inflammation.

Sensory sensitization refers to an increased responsiveness of sensory neurons to their normal input, or a response to previously sub-threshold stimuli. Both external or internal factors can initiate or exacerbate the heightened responsiveness. Tissue injury, inflammation, nerve damage, exposure to chemicals, and infections are examples of conditions that can result in sensory sensitization.

The airway mucosa, which lines the respiratory tract from the nasal cavity to the bronchi, is innervated by various types of sensory neurons that play pivotal roles in airway protection, regulation, and sensation. They can be divided broadly into thin unmyelinated (C fiber or nociceptors) or large myelinated neurons, each forming dense nerve plexuses under the epithelium. These neurons can have high or low threshold response to a given stimuli, respond to mechanical or chemical stimuli, adapt rapidly or slowly, have different ganglionic origin, or terminate at different central nuclei. They sense temperature, pressure, flow, and chemical composition of the surrounding environment providing feedback to their central terminals to initiate a reflex. Airway mucosal damage can lead to various respiratory complications.

Mucosal damage in the airway results in release of a cascade of inflammatory mediators, sensitizing nearby nociceptive neurons, making the airway hyperreactive. In a hyperreactive state, the airways are more responsive to a variety of stimuli, such as allergens, cold air, or exercise. Feedback loop of sensitization and Inflammation leads to persistent symptoms, even if the initial damaging agent is no longer present. Examples of airway hyperreactivity pathologies include asthma, chronic cough, and allergic rhinitis.

Sensitized neurons send a barrage of signals to CNS. This increased input leads to heightened activity in the central pathways by making synapses more potent or by reducing the efficacy of inhibitory mechanisms. This can create another feedback loop, where central sensitization further enhances peripheral sensitization by increasing the release of certain mediators or enhancing the responsiveness of peripheral nerves. As the brain not only processes sensations but also emotions and cognition, emotional state and peripheral damage can bidirectionally interact with each other through central-peripheral sensitization loops. In sleep apnea, local or systemic inflammation, airway hyperreactivity, gastrointestinal pathologies, viral infections, nasopharyngeal disease, exposure to air pollution, or hormonal fluctuations can result in airway sensory sensitization.

### Potential Causes for Sensory Sensitization in Sleep Apnea

### Inflammation

There is evidence that both systemic and upper airway inflammation are increased in obstructive OSA both in adults and pediatric population. Inflammatory markers are increased in sputum, exhaled breath condensate and nasal lavage specimens in OSA. Nasal mucosal inflammation is present in patients with sleep apnea even when no clinical sign of rhinitis is present. As described earlier, inflammation is a major contributor to sensory sensitization.

### Viral Infection

Role of viral infections in development of obstructive sleep apnea has been demonstrated in children. Sleep apnea patients are at increased risk of being diagnosed with a lower respiratory tract infection within one year of sleep apnea diagnosis while presence of OSA has been shown to exacerbate the symptoms of a viral infection.

### Fat Deposit

In obesity, fat deposition can impact lung volume and the caliber of airway, causing or exasperating respiratory pathologies. In addition to being a repository organ, adipose tissue is also an endocrine organ with role in energy homeostasis. It can secrete hormones, factors and protein signals called “adipokines” to perform its metabolic function. In obesity, dysfunction of adipose tissue can result in release of proinflammatory adipokines, induce cytokine storm, and activate intracellular pro-inflammatory signaling pathways. Levels of local and systemic inflammatory markers correlates with obesity and is elevated in obese patients versus non obese patients with airway inflammatory diseases. There is direct evidence linking adipose tissue dysfunction and airway hyperactivity.

### Nasopharyngeal Disease

Nasopharynx is the end point of mucociliary clearance of bacterial pathogens from the middle ear and paranasal sinuses and is normally colonized by both pathogenic and nonpathogenic bacteria in adult population. Potential respiratory pathogens gain entry to the host by colonizing the nasopharyngeal mucosal epithelium and normally remain in the airway for several months before being cleared by the immune response. In most cases these organisms are carried without causing clinical symptoms. Identifiable disease occurs in only a small percentage of those who are colonized. The presence of pathogens and the constant cycle of immune activation for pathogen removal can set the stage for sensory sensitization in the nasopharynx. Nasopharyngeal symptoms such as sneezing, mucus in the throat, and rhinorrhea have been observed in majority of OSA patients.

### Lymphoid Tissue Hypertrophy

Adenoid hypertrophy, the main cause of OSA in pediatric population, has been linked to childhood respiratory virus infection. Elevated concentrations of substance P and increased expression of its receptor, neurokinin 1, were detected in the upper airway lymphoid tissues of pediatric patients with OSA, suggesting that respiratory viral infection may have induced neuro-immunomodulatory changes within adenoid tissue. Nerve growth factor (NGF), a neurotropic factor, with a key role in bidirectional signaling between the nervous and immune systems, has been shown to be present in human lingual tonsils.

Lymphoid tissues, as reservoir of upper airway pathogens, can be the stage for multidirectional interaction between viral and bacterial pathogens, sensory neurons, and immune system.

### GI Related Pathologies

Gastroesophageal reflux disease (GERD) is a spectrum of disease that usually presents clinically with symptoms of heartburn and regurgitation, but can also present with extraesophageal manifestations, including chronic cough, chronic laryngitis, asthma, chest pain, postnasal drip, recurrent sinusitis, dysphonia and hoarseness. Two mechanisms have been proposed to explain laryngeal manifestations of GERD. The micro-aspiration theory postulates that there is direct acid-peptic injury to the larynx by esophagopharyngeal reflux, whereas the esophageal bronchial reflex theory proposes that pathologies in distal esophagus can induce laryngeal symptoms through a vagally mediated reflex. Either of the two theories can point to the potential for laryngeal afferent sensitization either through direct mucosal injury or one that is centrally propagated from other locations. Such interaction can explain the connection between GI symptoms and symptoms of sleep disorder breathing.

### Snoring

The theory of peripheral neurodegeneration in patients with obstructive sleep apnea was introduced in the late 1990s. Woodson saw focal degeneration of myelinated nerve fibers and axons of the soft palate tissue in OSA patients and snorers. Soft palate mucosa of OSA patients show an increased density in sensory nerve terminals, higher levels of inflammation, edema, neuropeptide, damaged axons, and neurotrophin upregulation compared with control subjects. Snoring has been postulated as a culprit in the sensory neural damage observed in the soft palate of OSA patients. Nerve injury and inflammation are known known culprits in sensory sensitization.

### Airway Hyperreactivity

Asthma and rhinitis are both airway hyperreactivity related diseases. A consistent body of scientific evidence supports the concept that rhinitis, rhinosinusitis, and asthma may be the expression of a common inflammatory disease, which manifests at different sites of the respiratory tract, at different times.

OSA and asthma have mutual impact on prevalence and symptom severity and exacerbations. Similarly, Rhinitis duration and severity is also associated with OSA. Significant number of patients with OSA present with irritable larynx, a laryngeal hyperreactivity syndrome, and chronic cough. Co-occurrence of OSA with other airway hyperreactivity diseases can point to involvement of air way inflammation in pathogenesis of OSA.

### Air Pollution

Air pollutants include toxic particles and gases emitted in large quantities from many different combustible materials. They also include particulate matter, ozone, and biological contaminants, such as viruses and bacteria, which can penetrate the human airway triggering airway inflammation. Air pollution exacerbate airway hyperreactivity diseases such as asthma, COPD, and rhinitis. Air pollution has been found to have a similar initiating and exacerbating effect in sleep apnea, pointing to inflammatory basis of the disease.

### Menopause

Sex hormones influence immune and nervous systems function. Airway hyperreactivity pathologies such as asthma, rhinitis, and chronic cough occur more prevalently in adult women than adult men and become more severe in women during pregnancy and menopause.

Like other airway hyperreactivity pathologies, the prevalence and severity of sleep apnea significantly increases during pregnancy and menopause in women. It has been shown that hormone fluctuations, more so than the absolute hormone levels, can impact peripheral sensory sensitivity in women, explaining symptom exacerbations and hypersensitivity development during periods of hormonal fluctuation such as menopause. Menopause can therefore increase the chances for sensory sensitization development.

### Sensory Innervation of the Upper Airway

The nose, sinuses and the nasopharynx are innervated by the trigeminal nerve. Anterior ethmoid nerve provides the sensory innervation to the anterior aspect of the nose, while the posterior nasal nerve provides sensory innervation to the posterior aspect. Superior alveolar nerve (SAN), a branch of the maxillary nerve, and its branches, anterior superior alveolar nerve (ASAN), middle superior alveolar nerve (MSAN), and posterior superior alveolar nerve (PSAN), innervate the maxillary sinus and the maxillary teeth and gingivae. Branches of SAN form the superior dental plexus within the mucosa of the maxillary sinus. Inferior alveolar nerve (IAN), a branch of the mandibular nerve, innervates gingivae on the lower jaw. Greater, lesser and nasopalatine nerves provide sensory innervation to the soft and hard palate. Pharyngeal and lesser palatine nerves branches of maxillary branch of the trigeminal nerve provide sensory innervation to the nasopharynx. Pharyngeal branch of glossopharyngeal nerve and pharyngeal branch of the vagus nerve innervate the oropharynx while superior laryngeal nerve (SLN), a branch of the vagus nerve provides sensory innervation to the laryngopharynx and the larynx above the vocal folds.

In contrast to nodose-derived vagal neurons that terminate centrally within the nucleus of solitary tract (NTS), jugular afferent fibers innervating the larynx, along with the glossopharyngeal neurons predominately terminate within trigeminal nuclei, especially within paratrigeminal nucleus, that acts to integrate the sensory inputs from the upper airway before relaying the information to other brain stem regions including the NTS. While mastication, breathing, and swallowing are controlled through central neural networks, peripheral sensory feedback, from the sensory neurons, plays and important role in modifying the generated patterns for protective purposes. The action of these neurons usually manifests in form of reflexes, the nature of which depends on the location, type, and intensity of the stimulus. Activation of upper airway sensory neurons can also initiate parasympathetic and sympathetic reflexes with cardiac and hemodynamic consequence. One of the most significant protective responses initiated by activation of upper airway sensory neurons is the mammalian diving response (MDR). MDR is an evolutionarily preserved, oxygen conserving, apneic reflex that is accompanied by overwhelming parasympathetic discharge that causes bradycardia combined with sympathetic activation resulting in hypertension and peripheral vasoconstriction. MDR is meant to protect mammals and diving terrestrial animals from filling their lungs with water during diving and to preserve oxygen for the brain during prolonged breath holds. MDR has variations depending on where it is initiated and the type and intensity of the stimuli. Stimulation of nasal and nasopharyngeal mucosa results in trigeminocardiac reflex (TCR) while Laryngeal chemoreflex (LCR) is initiated by chemical or mechanical irritation of the laryngeal mucosa.

Given the reflexogenic nature of the upper airway, the role of sensory neurons in initiating reflexes, and the intensity and consequential nature of the elicited reflexes, changes in the sensitivity of the sensory neurons can have significant implications for respiration. In fact, hyperreactivity of sensory neurons in other parts of the respiratory tract has been identified to be the cause or aggravator of respiratory diseases such as rhinitis, asthma, chronic obstructive pulmonary disease (COPD), and chronic cough. MDR-like reflexes have also been postulated to be the underlying cause of sudden infant death (SIDS) and sudden death in epilepsy. The following sections attempt to explain the potential impact of hypersensitivity of upper respiratory sensory neurons on the pathology and symptomology of sleep apnea.

### Role of Afferent Sensitization in Sleep Apnea Pathogenesis and Symptoms

### Role of the Vagus Nerve

### Upper Airway Muscle Function and Collapsibility

Upper airway muscles (UAMs), including laryngeal, pharyngeal, tongue and palatine muscles, receive their motor innervation from the trigeminal, facial, hypoglossal, glossopharyngeal and the vagus nerves. With hypoglossal and vagus nerves playing a dominant role. Both the hypoglossal and vagal motor activity is significantly influenced by airway vagal afferent input.

Considering that most of the occlusive episodes in OSA patients occur at the level of the oropharynx, laryngeal mechanoreceptors have an appropriate location for detecting sub atmospheric pressures and play an important role in maintenance of upper airway patency.

The pharyngeal and laryngeal mucosa are innervated by mechanosensitive myelinated fibers that form a dense nerve plexus under the epithelium. These neurons respond to both positive and negative pressures. Some are tonically active with respiration, and some are normally silent and are recruited once the intraluminal pressure deviates from eupneic conditions.

Activation of positive pressure sensing mechanoreceptors upon encountering larger than normal pressures results in airway closing reflexes such as swallowing. They close the airway by acting on UAMs. In response to negative pressures in the pharyngeal space, action of UAMs is increased by the activation of mechanoreceptors. Whether upper airway muscle activation is facilitated by direct activation of negative pressure sensing neurons, their disinhibition, or inhibition of positive pressure neurons is not fully clear in humans. Regardless of exact mechanism the net effect is the activation of upper airway abductor muscles and prolongation of inspiratory and expiratory timing. Upon activation of these neurons, or inhibition of positive pressure sensors, the soft palate lowers to open the retropalatal space and the tongue stiffens and moves anteriorly to maintain the retroglossal patency and expose the larynx.

Normally, the threshold for activation of the mechanoreceptors responding to positive or negative pressure results in well delineated ranges of positive and negative pressures within which protective reflexes are initiated. In a state of hypersensitivity, the threshold for activation of mechanoreceptors is lowered. This would bring the threshold of positive-pressure neurons closer to atmospheric pressures. As activation of positive pressure sensors has an inhibitory impact on UAMs, small positive pressures, such as those generated by normal expiration, can result in inhibition of UAMs and increase airway resistance. Higher air velocity due to increased airway resistance further increases the firing of afferents compounding the effect. Majority of obstructive events in OSA patients occur during positive or close to atmospheric pressures.

Due to sensitization, C fibers, normally not responsive to mechanical stimuli can now fire spontaneously, or in response to pressure, adding to the chorus of neurons than normally fire with pressure and potentially those that are normally silent but are now firing. As a result, a large background “noise” is produced. For negative pressure sensors to be activated, the pressure should be negative enough to overcome the noise. This would mean that a larger than normal negative pressure would be required to activate or disinhibit negative pressure mechanosensors, making the mucosa practically insensitive to philologically relevant negative pressures.

In OSA, airway closure in response to positive pressures happen because the threshold for initiation of airway closing defensive reflexes has moved lower, close to atmospheric pressures. Negative pressure obstructions happen because airway opening, negative pressure reflexes, now require much lower negative pressures to activate. Therefore, the airway collapses before those levels of negative pressure are reached, making the mucosa practically insensitive to negative pressures.

### Dysphagia

Stimulation of SLN inhibits respiration and causes an expiratory apneic state by inhibiting inspiration and prolonging expiration during swallowing. This is in line with the fact that swallows are evolved to happen during expiration as swallowing during inspiration increases the risk of aspiration. Upon activation of SLN afferents, for example by activation of mechanoreceptors or chemoreceptors, breathing ceases and the expiration is extended to allow for swallows to occur safely.

In state of hypersensitization due to spontaneous firing and recruitment of silent neurons, a barrage of sensory information, generated by airway mucosal afferents, is constantly transmitted to the brainstem that can result in repetitive apneas and cessation of breathing with every swallow or even in the absence of a specific stimuli. While the pattern generators that integrate sensory information for airway defense are in the brainstem, the cerebral cortex also plays a role in integrating sensory information and coordination of defensive reflexes. In fact, peripheral afferent projections do not terminate solely in the brainstem, but instead continue to ascend toward the thalamus and ultimately to cortical regions. The term “gating” refers to the ability of the cerebral cortex to include or exclude sensory information based on its relative redundancy or relevance. Sensory “gating out” of information is defined as reduced neural activity occurring with increased stimulus redundancy. In a healthy subject, due to the importance of airway protective reflexes in maintaining airway patency, oropharyngeal sensory information related to swallowing or coughing are not gated out. However, it is possible, that in case of hyperactive sensory input, and to provide necessary oxygen for daily functional activities and maintain homeostasis, the highly redundant sensory information is gated out. This theory is consistent with prevalence of comorbid dysphagia in patients with OSA. Gating out of pharyngeal and laryngeal sensory information to maintain airway patency comes at the cost of swallowing disorder.

### Impact of Sleep

While during wakefulness the redundant sensory information is gated out, this does not seem to happen during sleep in OSA patients. One possible explanation could be the impact of thalamic neuropeptides in the gating out process and their fluctuation between wake and sleep. Orexins, also known as hypocretins, are neuropeptides that are exclusively expressed by neurons in the thalamus. Although originally recognized as regulators of feeding behavior, orexins are now mainly regarded as key modulators of the sleep/wakefulness cycle.

In rats, orexin A has been shown to inhibit reflex swallowing induced by stimulation of SLN. Orexin has also been shown to play a role in the thalamus somatosensory gating in rats. In case of trigeminal hypersensitivity, orexin has been shown to play an inhibitory role in nociception. Orexin neurons discharge during active waking, when muscle tone is high in association with movement, decrease discharge during quiet waking in absence of movement, and virtually cease firing during sleep, when muscle tone is low or absent. During rapid eye movement (REM) sleep, they remain relatively silent in association with postural muscle atonia and most often despite phasic muscular twitches. They increase firing before the end of REM sleep in preparation for wakefulness and the return of muscle tone.

If like rats, in humans, orexin plays a role in gating out the barrage of sensory information produced by hyperactive laryngeal neurons, its decreased levels during sleep would disinhibit the sensory overload. Lower levels of orexin, combined with decreased muscle tone can potentially result in hypersensitized neurons' aberrant signaling to result in repetitive apneas and airway closures because of mechanosensitive neurons firing in response to normal intraluminal pressures.

Physiological orexin is lower in men than in women which in addition to other physiological and anatomical factors can potentially contribute to higher rates of OSA in male. However, high levels of orexin have also been shown to result in sleep disorders and panic attacks. Women sex hormone, 17-β estradiol, has been shown to have an inhibitory impact on orexinergic neurons. Levels of orexin has been shown to rise in menopausal wome. Disinhibition of orexinergic neurons, by diminished levels of 17-β estradiol, and higher orexin levels in menopause, can provide a potential explanation for OSA onset in menopausal women.

### Central Apnea, Bradycardia, and Hypertension

Chemical irritation or mechanical stimulation of nasopharyngeal or laryngeal mucosa has been shown to induce apnea, bradycardia, and hypertension in form of MDR. As the increased sensitivity of the sensory neurons, the threshold for initiation of such extreme reflexes decreases to a point where normal or low intensity stimuli can cause intense sympathetic and parasympathetic activation. Indeed, apnea, bradycardia and hypertension are all hallmarks of sleep apnea.

Activation of transient receptor potential (TRP) channels, such as TRPA1 and TRPV1, are thought to be one of the mechanisms of inflammation induced hypersensitivity. Hypoxia and generation of reactive oxygen species are also shown to sensitize these channels. Therefore, inflammation induced sensitization, MDR activation, apnea, hypoxia, followed by hypoxia induced inflammation can result in a self-sustaining feedback loop defining the progressive nature of OSA.

The central apnea produced by the MDR reflex can also have detrimental impact on airway collapsibility. Pulmonary slowly adapting receptors (SAR) have an inhibitory impact on upper airway muscle function to prevent lung overinflation. An apneic event occurring at peak inspiration, withholding deflation of a fully inflated lung, can result in expiratory effort against an airway closed by UAMs inhibition. The airway will stay closed until SAR signaling is diminished, through slow adaptation, or the activation of respiratory muscles overcome the obstruction.

### Arousal

While MDR is an exaggerated protective reflex, arousal from sleep is a protective reflex to prevent asphyxiation due to apnea. Stimulation of trigeminal and laryngeal afferents have been shown to result in dose-dependent arousals.

Airway defensive reflexes, specifically swallowing or glottic adduction, influence cardiac autonomic activity and cortical arousal from sleep. Swallows trigger rapid, robust, and patterned tachycardia conserved across wake, sleep, and arousal states. Multiple swallows increase the magnitude of tachycardia via temporal summation, and blood pressure increases. During sleep, swallows are overwhelmingly associated with arousal, duration of which increases with swallow incidence. Lack of arousal in infants and newborns have been postulated to be the cause of SIDS. It can then be inferred that hypersensitivity of airway defensive reflexes, especially swallowing, will increase the incidence and intensity of arousals such as those observed in OSA.

### Loop Gain

The chemo and baro receptors in the carotid body and carotid sinus, together called peripheral chemoreceptors, are supplied by the carotid sinus nerve (CSN), a branch of glossopharyngeal nerve. Recent anatomical investigations have shown that SLN connects with CSN and innervates the carotid sinus and carotid bodies. It is then reasonable to believe that sensitization of SLN afferents can directly propagate the sensitization to peripheral chemoreceptors. Peripheral chemoreceptor sensitization has been observed in OSA patients.

Blunted ventilation, because of MDR induced central apnea or apnea caused by an obstructive event caused by airway defensive reflexes, can result in pent-up CO₂ and depleted oxygen reserves. In response, a proportionally large ventilatory effort in form of hyperventilation is generated to regain equilibrium. As hyperventilation elevates the oxygen levels, the hypersensitive peripheral chemoreceptors can initiate hyperoxic reflexes at oxygen levels below or within eupneic levels, swinging the pendulum. This temporary blunting of ventilation together with peripheral chemoreceptor sensitization and their exaggerated hyperoxic response can result in ventilation instability and increased loop gain.

### Depression and Anxiety

After jugular ganglia, the primary central relay point for the laryngeal afferent input is paratrigeminal nucleus (Pa5). Pa5 has been shown to send projections to submedius thalamic nucleus (SubM) and from there to ventrolateral orbital cortex (VLO) and periaqueductal gray (PAG), regions known to regulate the endogenous analgesia system. The SubM-VLO has been shown to have descending inhibition on nociceptive somatosensory input resulting in analgesia and anti-anxiety and anti-depressive effects. The VLO can significantly decrease in cases of afferent hypersensitivity such as those seen in chronic nerve injury. Therefore, hypersensitivity of jugular vagal afferents and their subsequent inhibition of the endogenous analgesia system can explain the anxiety and depression symptoms frequently observed in patients with sleep apnea.

### Role of the Trigeminal Nerve

The afferents innervating the nose, sinuses and the nasopharynx are capable of measuring airflow, temperature, and chemical composition of the inhaled air. Trigeminal afferents innervating the nose and the nasopharynx are the first defense measure against inhaled bacteria, pathogens, and hazardous chemicals. The afferent input from the nasal receptors, are integrated centrally into respiratory centers along with other upper airway afferent input and can impact breathing patterns.

Upon identification of a threat, nasal afferents initiate a series of protective reflexes to narrow the airway and prevent further exposure. They initiate sympathetic and parasympathetic reflexes that cause vasodilation, mucus secretion, and sneezing to eliminate the offending object. Nasal (trigeminal) hyperreactivity manifests in form of rhinitis, both allergic and non-allergic, and common cold. Patients with rhinitis are at a higher risk of developing sleep disordered breathing. Nasal and nasopharyngeal hyperreactivity can also result in postnasal drip, spreading the inflammatory mediators further down in the pharynx and larynx.

### Airway Resistance

Patients with nasal hyperreactivity, such as rhinitis have higher levels of nasal resistance. Similarly, inflammation of nasopharyngeal mucosa can impact airway size and resistance. It has also been shown that the degree of nasal flow limitation correlates with the apnoea/hypopnoea index in OSA patients.

Nasopharynx has attracted attention in pediatric OSA due to adenotonsillar enlargement. Nasopharynx is the collection vessel for the inhaled viral and bacterial pathogens. Considering the role of pathogens in sensory sensitization, nasopharyngeal mucosa is vulnerable to inflammation and sensory sensitization.

Inflammation in the nasopharynx can result in reduction of airway diameter at the nasopharyngeal level. According to Bernoulli's principle, reduced diameter can result in increased air flow velocity and reduction of static pressure. Larger negative pressures can the result in retropalatal collapse. Indeed, nasopharyngeal inflammation has been shown to be a contributor to increased retropalatal mechanical loads in OSA patients.

### Impact on Uams

Trigeminal nerve does not have significant direct contribution to innervation of UAMs; however, it can impact their activity through shared central integration with vagal afferents. Bypassing nasal breathing results in disordered breathing pointing to the role of nasal receptors in modulating upper airway patency. In humans nose breathing augments the activity of UAM.

Evidence suggests that trigeminal afferents innervating the nose measure air temperature and CO₂ concentration and their differential between anterior and posterior aspects of nasal cavity to estimate the level of flow and its inspiratory versus expiratory nature. TRPA1 has been shown to play an important role in temperature and CO₂ sensing in the nasal cavity. While eupneic breathing would not initiate a protective response, out of normal temperature and CO₂ differentials between inspired and expired air can impact airway resistance and initiate protective reflexes. Sensitization of trigeminal nasal afferents, especially C fibers and their TRP channels, their spontaneous firing and lowered threshold, and their overestimation of CO₂ and temperature differentials, can result in initiating airway protective reflexes that result in increased airway resistance and make the nose insensitive to CO₂ drop due to an ensuing apneic event.

### Otologic Symptoms

Input from trigeminal afferents innervating the nasopharynx impact the opening and closing of the eustachian tube. While input from these afferents can impact respiration by instantiating motor responses in UAMs through central connection with the vagus nerve, their input can also facilitate opening or closing of the eustachian tube by motor reflex on tensor veli palatini muscle through trigeminal central relays. Afferents fire when the measured nasopharyngeal-auditory tube CO₂ level differential falls outside a threshold. Lowered threshold of nasopharyngeal afferents can result in unnecessary closing of the eustachian tube, causing eustachian tube dysfunction and its associated symptoms. Increased background somatosensory noise, due to spontaneously firing C fibers, and the eustachian tube dysfunction can potentially explain ear symptoms such as fullness, tinnitus, Meniere's disease, and hearing loss in a subset of patients with OSA.

### Apnea, Bradycardia, Hypertension

Nasal and especially nasopharyngeal stimulation can initiate MDR resulting in apnea bradycardia, hypertension, and gastric mobility. The impact of sensory sensitization of trigeminal afferents in initiation of parasympathetic and sympathetic activation is like those of laryngeal C fibers. In a state of hypersensitivity, trigeminal C fiber afferents can induce MDR like reflexes with its respiratory, cardiovascular, and hemodynamic consequences.

### Bruxism

Both hard and soft palate have a dense sensory innervation including mechanoreceptors and C fibers. Palatal mechanoreceptors work with lingual and gingival mechanoreceptors to determine the size, texture and hardness of a bolus and adjust mastication force and rhythm. Palatal afferents have direct impact on activation of jaw-closing, jaw-opening, and tongue muscles. Mechanical stimulation of soft palate has been shown to decelerate breathing. Lesser palatine nerve (LPN) innervates the soft palate while the greater palatine (GPN) and nasopalatine (NPN) nerves innervate the posterior and anterior hard palate, respectively. These nerves also innervate part of the nose and nasopharynx. Inflammation in the nose, nasopharynx, or soft palate can result in palatine nerve sensitization. The lowered threshold of the mechanoreceptors and the aberrant firing of the C fibers of palatal sensory afferents can slow down respiration, initiate fictive mastication, as seen in bruxism, and impact tongue muscle activity. OSA is one of the most frequent risk factors for sleep bruxism in the adult population. It has been shown that most of sleep bruxing episodes co-occur with swallowing, pointing to interconnectedness of airway protective reflexes and the action of jaw muscles, probably coordinated through the central connection between trigeminal and laryngeal vagal afferents. Interestingly, other airway hyperactivity syndromes such as rhinitis, rhinosinusitis, and asthma have also been shown to be associated with bruxism, pointing to inflammation as a potential culprit.

### Periodontitis

The sensory innervation to the gums is provided by the maxillary branch of the trigeminal nerve on the maxilla and the mandibular branch on the mandible. The sensory innervation of the trigeminal nerve includes both C fibers and mechanoreceptors. Inflammation in the nose and nasopharynx can result in sensitization produced in one maxillary branch to propagate to other branches of the trigeminal nerve. As mandibular and maxillary fibers are in proximity in the oral mucosa, especially in the palate, sensitization and inflammation can spread easily. ASAN, MSAN, PSAN, and IAN innervate the periodontal tissue, and their sensitization can result in local inflammation and vice versa. Abundance of bacteria in the mouth can also initiate or exacerbate the inflammation causing a progressive disease. In addition to the sensory innervation, gingival and periodontal tissue receive their parasympathetic innervation from the glossopharyngeal and trigeminal nerves through the otic and sphenopalatine ganglia, respectively. Hyperreactivity of the sensory neurons innervating the gingival and periodontal mucosa can initiate a parasympathetic reflex, resulting in vasodilation, plasma extravasation, and edema within gingival and periodontal tissues, all hallmarks of gingivitis and periodontitis disease. The close contact and shared pathway of sensory neurons that innervate the gums and palate with those that innervate the airway can explain the high incidence of periodontitis in patients with OSA. This connection also explains the relationship between periodontitis, heart failure and hypertension.

### Mouth Breathing

In addition to providing sensory innervation to the soft palate, LPN carries motor fibers from the facial nerve to the soft palate. Stimulation of LPN can elevate the nasal aspect of the soft palate contributing to velopharyngeal closure. Sensitization of both vagal and trigeminal nerves have been shown to increase facial nerve activity. If sensitized, LPN can influence soft palate movement, increasing its potential to elevate, making a retropalatal obstruction more likely. Additionally, activation of levator veli palatini (LVP), that receives motor innervation through LPN, plays an important role in switching route of breathing. Activation of LVP has been shown to switch breathing from nasal to oral route. This could explain the prevalence of mouth breathing in patients with sleep apnea.

### Disease Progression

Two self-reinforcing loops underlie the progressive nature of sleep apnea. First is the impact of hypoxia on increasing sensory sensitization, and second is the bidirectional relationship between peripheral and central sensitization. As sensitization increases, so does the frequency of obstructive events and their associated hypoxia, increasing inflammation and sensitization. Sensitization at the central terminals of sensitized neurons initiates central sensitization which will in turn reinforce the sensitization in the location of sensitization origin and broadcast it to its other peripheral and central connections, expanding the reach and consequences of an initially limited peripheral event. Intermittent hypoxia can also directly result in central sensitization, opening another avenue for sensitization propagation. These self-reinforcing cycles result in far reaching symptoms of sleep apnea and its progressive nature.

### SUMMARY OF THE INVENTION

The upper airway is constantly monitored by an array of sensors innervated by the trigeminal and the vagus nerves. These sensory neurons, monitor the environment and potentiate reflexes in response to a stimuli. The reflexes are in form of local release of neuropeptides to activate immune cells or act on blood vessels, potentiate centrally mediated motor response to upper airway muscles, or potentiate centrally mediated parasympathetic and sympathetic responses. The outcome of this coordinated sensing and response feedback loop is the adjustment of upper airway resistance, patency, and coordination of functions such as breathing, eating, drinking, or speaking. The motor response potentiated from sensory neurons input activate upper airway muscles, to move structures that act as valves, in and out of the airway to close the airway or create variations of openings for different functions such as mastication, swallowing, and breathing. These muscular structures, include soft palate, tensor veli palatini muscle, tongue, and epiglottis. The autonomic reflex acts on the mucosa and the arteries and veins to increase or decrease the mucosal thickness which also impacts the airway resistance and patency. The autonomic reflex also activates secretory cells to release mucus to wash out an offending agent such as an allergen or lubricate the airway. Of note, the nasal valve and nasal turbinates can increase their size due to the action of the autonomic reflex to modify airway resistance. In some instances, the parasympathetic and sympathetic fibers that innervate the mucosa of the airway reach their target tissue by merging with the same nerve that carries the sensory fibers. In other cases, the parasympathetic and sympathetic innervation is provided by a different nerve. Therefore, the parasympathetic reflex can be treated at the same time as treating the sensory nerve or is treated in a separate procedure performed of a separate nerve.

The motor response activates muscles to open or close the airway. The autonomic reflex acts on the mucosa, arteries, and veins to modulate the mucosal thickness which impacts airway resistance and patency by changing airway caliber. The autonomic reflex result in mucus secretion and vasodilation which also impact airway resistance.

The activity of sensory neurons has a significant impact on the function and patency of the upper airway and their dysfunction can result in serious conditions such as sleep apnea, rhinitis, bruxism, ear pathologies, jaw movement pathologies, and chronic cough. The current disclosure teaches methods to treat diseases affecting the airway, by either treating the malfunctioning sensory nerve or treating the mucosa that a given sensory nerve innervates. Treating the mucosal tissue is intended to treat sensory neuron receptors or the parasympathetic and sympathetic effector cells that are resident in the tissue. It is also possible to treat a disease by treating the nerve that carries the autonomic innervation to the mucosal tissue that is innervated by a given sensory nerve to interrupt the sensory-autonomic response feedback loop.

The choice of the target, whether the nerve itself or the tissue the nerve inntervates, is determined by the symptoms and their patterns in a patient. Matching the location where the symptoms occur to a sensory nerve's pattern of innervation and its motor and autonomic reflex arcs can identify the tissue or the nerve(s) to be treated. The mode of treatment also depends on the accessibility of the nerve and tissues. For example, some nerves are accessible through non-invasive methods while others are not. While, directly treating the nerve allows for treatment in one or multiple discreet locations, treating the tissue might require larger treatment areas but both the sensory and autonomic pathways to a given tissue can be treated.

Embodiments provided in this disclosure are examples of treatment options to affect the sensory-response loops that govern the upper airway function and are by no means exhaustive. Other variations of targets to treat, diseases to treat or modes of treatment can be inferred.

In one embodiment, treating a disease comprises methods to modify the activity of a sensory nerve. Example of such strategies include but are not limited to; method used to silence, block, ablate, resect, reduce the activity, reduce the frequency of firing, reduce the amplitude of firing, modify the threshold of firing, modify the receptors, block the receptors or in general reduce the hyperreactivity of a nerve. In another embodiment, treating a nerve comprises treating the nerve itself or any of its branches or ganglia.

In another embodiment, treating a disease comprises treating the tissue that a given sensory nerve, or its branches, innervate. In another embodiment, treating the tissue a nerve innervates include treating at least a portion of sensory, parasympathetic, or sympathetic fibers that are embedded within the tissue. In yet another embodiment, treating the tissue a nerve innervates, comprises treating at least a portion of the resident cells within the tissue. Examples of resident cells include, but are not limited to, secretory cells, goblet cells, basal cells, epithelial cells, glands, ciliary cells, solitary chemosensory cells, lymphocytes, or lymphoid follicle. In one embodiment, treating the tissue a nerve innervates comprises treating at least one blood vessel running within that tissue.

In yet another embodiment, treating a disease comprises treating the nerve(s) or ganglia that provide autonomic fibers to the tissue the sensory nerve innervates. In another embodiment both the sensory and autonomic pathways to a tissue is treated to treat a disease.

In one embodiment, at least a portion of a pharyngeal nerve (PhN), a lesser palatine nerve (LPN), a greater palatine nerve (GPN), a nasopalatine nerve (NPN), a pharyngeal branch of the glossopharyngeal nerve (PhIX), a pharyngeal branch of the vagus nerve (PhX), and a superior laryngeal nerve (SLN) is treated to treat a disease.

In another embodiment, at least a portion of the mucosa innervated by at least a PhN, LPN, GPN, NPN, PhIX, PhX, and SLN, is treated to treat a disease.

In one embodiment, hyperreactivity of these nerves is the underlying cause of the diseases to be treated. In one embodiment, hyperreactivity of these nerves is due to the malfunctioning of their receptors. In another embodiment, the hyperreactivity of these nerves is a result of local or systemic inflammation, nerve injury, tissue damage, allergy, immune disorder, exposure to environmental pollution, hormonal changes, viral or bacterial infection, obesity, or is related to a central nervous system (CNS) disorder. In one embodiment, these nerves are the original site of the inflammatory disease and neural hyperactivity, while in another embodiment, the hyperreactivity is propagated to them from another peripheral or central location.

In one embodiment, at least one of PhN, LPN, PhIX, PhX, and SLN, or a portion of the tissue they innervate, is treated to treat or reduce symptoms associated with sleep breathing disorders (SBD), a class of disorders that include but not limited to upper airway resistance syndrome, central apnea, obstructive sleep apnea (OSA), mixed apnea, or snoring.

In another embodiment, at least one of LPN, PhIX, PhX, and SLN, or the a portion of tissue they innervate, is treated to treat or reduce the symptoms associated with diseases that include but are not limited to; SBD, heart failure, cardiovascular disease, hypertension, temporomandibular joint disorder (TMD), renal disease, pulmonary disease, diabetes, obesity, dyslipidemia, thyroid disease such as goiter and hypothyroidism, neuropsychiatric diseases, metabolic disease, neuroimmune disease, dysphagia, swallowing disorders, gastrointestinal disorders, lower respiratory tract disorders, cough, voice disturbances, otologic conditions such as eustachian tube dysfunction, tinnitus, ear fullness, Meniere's disease, hearing loss, bruxism, gingival and periodontal inflammatory conditions, mouth breathing, anxiety, depression, daytime sleepiness, wheezing, nasopharyngeal symptoms such as postnasal drip, water in the throat, throat dryness, rhinorrhea, nasal obstruction, lymphoid tissue hypertrophy, laryngeal hyperreactivity syndrome, or other disorders that are directly or indirectly associated with hyperreactivity of the aforementioned nerves.

In another embodiment, at least one of LPN, PhIX, PhX, and SLN, or a portion of tissue they innervate, is treated to treat heart failure or symptoms associated with it, including, and not limited, to bradycardia, arrhythmia, SBD, hypertension, shortness of breath, fatigue, and weakness, swelling in the legs, ankles and feet, rapid or irregular heartbeat, wheezing, cough, fluid buildup, nausea and lack of appetite, difficulty concentrating or decreased alertness.

In one embodiment, at least one of PhN, and LPN, or a portion of tissue they innervate, is treated to treat or reduce symptoms of otologic conditions such as but not limited to eustachian tube dysfunction, tinnitus, ear fullness, Meniere's disease, hearing loss, nasopharyngeal symptoms such as mucosal inflammation, coughing, sneezing, postnasal drip, water in the throat, throat dryness, rhinorrhea, sore throat, sneezing, lymphoid tissue inflammation such as adenoid hypertrophy, or tonsillar hypertrophy. Examples of instruments which may be used to treat regions of the nasopharynx mucosa such as the PhN, according to the description herein, are described in further detail in, for example, U.S. Pat. No. 9,801,752 and U.S. Pub. 2021/0052318. Each of these references is incorporated herein by reference and for any purpose. However, this embodiment treatment may be utilized in combination with other treatments described herein in any combination or order of steps.

In another embodiment, at least one of LPN, GPN, and NPN is treated to treat or reduce symptoms of sleep-related sleep disorders such as sleep bruxism, awake bruxism, orofacial muscle pain, myalgia (muscle pain), endinosis or enthesopathy, myositis, spasm, jaw movement disorders such as orofacial/oromandibular dyskinesia and dystonia, oral mucosal inflammatory disorders, temporomandibular joint disease, palatal inflammatory disorders such as palatal torus, abscess or cyst, gingival or periodontal inflammatory diseases such as periodontitis and gingivitis.

In another embodiment, at least one of SAN, ASAN, MSAN, PSAN, or IAN is treated to treat or reduce symptoms of sleep-related sleep disorders such as sleep bruxism, awake bruxism, orofacial muscle pain, myalgia (muscle pain), endinosis or enthesopathy, myositis, spasm, jaw movement disorders such as orofacial/oromandibular dyskinesia and dystonia, oral mucosal inflammatory disorders, temporomandibular joint disease, palatal inflammatory disorders such as palatal torus, abscess or cyst, gingival or periodontal inflammatory diseases such as periodontitis and gingivitis.

In another embodiment, to reduce the impact of malfunctioning of a sensory nerve, the nerve(s) or ganglia that provide autonomic fibers to the sensory nerve, or the tissue in innervates, is treated to reduce at least one symptom of a disease.

In one embodiment, SLN is treated anywhere along its course. In one embodiment, the internal branch of SLN (iSLN) is treated. In one embodiment, iSLN is treated at its point of entry into the thyroid membrane, or anywhere along its course on the oropharyngeal or laryngopharyngeal mucosa. In one embodiment, at least a portion of oropharyngeal or laryngopharyngeal mucosa is treated to treat diseases associated with hyperreactivity of SLN.

In one embodiment, treatment of PhN is conducted at any point along its course from the sphenopalatine ganglion, through sphenoid sinus, along the palatovaginal canal, at palatovaginal foramen, on the mucosa of nasopharynx, around the auditory tube orifice, or within the auditory tube. In another embodiment, at least a portion of the mucosa of the nasopharynx or auditory tube is treated.

In another embodiment, treatment of LPN is conducted at any point along its course from the sphenopalatine ganglion, through the palatine canal, at lesser palatine foramina, or along its course in the soft palate. In a preferred embodiment, LPN is treated at lesser palatine foramina. In another embodiment, at least a portion of mucosa of the soft palate is treated.

In another embodiment, GPN is treated at any point along its course from the sphenopalatine ganglion, through the palatine canal, at greater palatine foramina, or along its course in the hard palate. In another embodiment at least a portion of mucosa of the hard palate is treated.

In one embodiment, NPN is treated at any point along its course from the sphenopalatine ganglion, in the nasal cavity, along the nasal septum, nasal floor, at the incisive canal, or along its course in the hard palate. In one embodiment, to treat NPN hyperreactivity related diseases, at least a portion of mucosa of hard palate is treated.

In one embodiment, to treat diseases related to PhN, LPN, GPN, NPN, greater petrosal nerve (GPeN), that provides autonomic fibers to these nerves is treated. In one embodiment, GpeN is treated anywhere along its course or its ganglia.

In one embodiment, SAN, ASAN, MSAN, PSAN are treated anywhere along their course from the maxillary nerve, through the maxillary sinus on along their course on the gingivae. In one embodiment to treat one of SAN, ASAN, MSAN, PSAN, sphenopalatine ganglion is treated. In another embodiment to treat at least one of SAN, ASAN, MSAN, PSAN, GpeN is treated.

In one embodiment, the target nerve is treated with a chemical or pharmaceutical agent. In one embodiment, the chemical or pharmaceutical agent is intended to treat malfunctioning, such as reducing hyperreactivity, of the target nerves or its associated receptors. In another embodiment, the chemical or pharmaceutical agent is intended to modify the parasympathetic or sympathetic response of the malfunctioning, or hyperactive sensory nerve.

In one example, the chemical or pharmaceutical agent is injected or otherwise applied to the proximity of the sensory nerve along its course or in the tissue the nerve innervates. In another example, the chemical or pharmaceutical agent is injected or otherwise applied to the proximity of the nerve(s) that carry the parasympathetic or sympathetic fibers to the tissue the sensory nerve innervates. In another embodiment, the chemical or pharmaceutical agent is applied on the tissue the sensory nerve innervates. In one embodiment, the chemical or pharmaceutical agent is formulated with other ingredients to enhance its activity, stability, or deliverability. Formulation ingredients can include but are not limited to polymers, viscosity or osmolarity adjustors, stabilizers, humectants, pressurized gas, or mucoadhesive agents.

In one embodiment, the chemical or pharmaceutical agent is in a powder form and is inhaled through the nose or the mouth. In yet another example, the chemical or pharmaceutical agent is in liquid form and is sprayed on or otherwise applied through intranasal or intraoral route. In yet another embodiment, the chemical or pharmaceutical agent is directly applied on the target tissue using an applicator. In yet another embodiment, the chemical or pharmaceutical agent is in liquid form and is applied by swishing the liquid in the nose or mouth such as nasal rinse, oral rinse, mouth rinse, or mouth wash. In another embodiment, the chemical or therapeutic agent is embedded within a depot and placed, injected, or implanted in the proximity of the target nerve, or in the tissue it innervates, to achieve sustained release and extend the therapeutic duration. Examples of depots includes but is not limited to liposomes, microparticles, microspheres, polymeric films, in vivo solidifying polymers, in-situ crosslinkable polymers, injectable polymers, degradable or nondegradable polymers, or pumps such as osmotic pumps.

Examples of therapeutics agents include those that act on the sensory nerve receptors such as C fibers, cough receptors, mechanoreceptors, and chemoreceptors to inhibit or reduce their function. Examples of pharmaceutical agents are, but not limited to, TRP channel inhibitors such TRPA1 and TRPV1 antagonist, substance P antagonist, CGRP antagonist, P2×3 antagonist, P2×2 antagonists, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin or other neurotoxin agents, anesthetics, analgesics, corticosteroids, or combination thereof. Other examples of therapeutics agents include those that target the parasympathetic or sympathetic pathways of the hyperreactive nerve. Examples of agents impacting parasympathetic pathway include, but is not limited to, VIP antagonist, muscarinic antagonists, nicotinic antagonists, anticholinergic agents. In another embodiment, the pharmaceutical agent is used to increase the sympathetic activity in the target tissue that is innervated by the hyperreactive sensory neuron. Examples of such pharmaceutical agents include, but is not limited to, beta blockers, beta-2 (β2) agonists and alpha-2 (α2) agonists. Examples of chemicals include, but not limited, to glycerol, phenol, or ethyl alcohol. In another embodiment, the pharmaceutical treatment comprises a combination of agents impacting multiple receptors or enhancing the action on one receptor.

In another embodiment, the sensory nerve, its autonomic counterpart, or at least a portion of tissue it innervates, is treated using physical or mechanical methods. In one example, the nerve or the tissues it innervates, are surgically sectioned or resected.

In another embodiment, mechanical pressure is applied on the nerve or at least a portion of the tissue it innervates to reduce the activity of the said nerve, its terminal branches, or receptors. In one embodiment, a device with a proximal end, a distal end and an elongated shaft is used to apply pressure on the nerve or its target tissue. In one embodiment, the distal end of the device is expandable, and the device is in a collapsed state during navigation to the target nerve or tissue, once it arrives at the proximity of the target, the distal end is expanded to apply desired level of pressure on the target location. In one embodiment, an expandable balloon is used to treat the nerve or a portion of the tissue it innervates.

In another embodiment, a physical barrier is applied on at least a portion of the tissue the target nerve innervates to protect it from the stimuli or forces that activate the nerve or its receptors. In one embodiment the physical barrier is made of plastic, degradable or nondegradable polymers, in-situ cross-linkable polymers, hydrogels, or metals. In one embodiment, the physical barrier is preformed to match the patient and fit the anatomy of the tissue it is intended to protect. In one embodiment the physical barrier is in one shape during delivery and is capable to change shape to fit the anatomy it is intended to treat once it reaches target location.

In another embodiment, the target nerve, or a portion of the tissue it innervates, is treated using energy. Examples of energy modalities include but are not limited to radiofrequency energy, pulsed radiofrequency energy, laser, ultrasonic energy, thermal energy (either heat or extreme cold), electrical energy, electromagnetic energy, or light. In one embodiment, treatment using energy is performed by using an effector device. The device contemplated here comprises a proximal end connected to a distal end by an elongated shaft. The proximal end is to be held and manipulated by an operator (e.g., a surgeon) to navigate the distal end to the proximity of the target. The distal end of the device comprises an effector component. The distal end of the device is positioned on or in proximity of the target nerve, or at least a portion of the tissue it innervates before the effector is activated. Once treatment is completed, the effector is de-activated, and the device is navigated away from the target.

In one embodiment the distal end of the device used for treatment with energy is capable of changing shape once arriving at target tissue. For example, the distal end can have a collapsed configuration during navigation to the target. Once at the target, the distal end can be made to change configuration from collapsed to expanded form to provide maximal contact with the target. Once the treatment is completed the distal end of the treatment device can once again be made to change from an expanded configuration to a collapsed configuration, to facilitate navigation away from the target. In one embodiment, the distal end of the treatment device is an expandable balloon. In another embodiment, treatment device has an illumination, magnification, or navigation component.

In one embodiment, treatment with energy comprises ablating the nerve or at least a portion of the tissue the nerve innervates. In another embodiment, treatment with energy comprises stimulating the nerve in a manner that counters its hyperreactivity. In yet another embodiment, treatment with energy comprises reducing the conduction of the nerve.

In one embodiment, the nerve, or the tissue it innervates is treated by extreme cold. In another embodiment, the nerve, or a portion of the tissue it innervates is treated at temperatures in the range of, e.g., −20° C. to −100° C.

In one embodiment, the cryogenic treatment is conducted using a device comprising a proximal end, a distal end and an elongated shaft. In one embodiment the cryogenic device uses the Joules-Thomson (JT) effect to produce the ultra-low temperature. In such a configuration the high-pressure refrigerant gas or liquid is injected through an internal lumen to the distal end of the device such that when the gas leaves the internal lumen at the proximity of the distal end, it expands into an outer lumen causing a drop in temperature. The exhaust gas is then released back to the environment. In another embodiment, the refrigerant gas used to produce the JT effect can also be used to expand the distal end of the device when the distal end is designed to be expandable.

In another embodiment, the cryogenic gas or liquid is directly sprayed on the target nerve or at least a portion of the tissue it innervates. In this embodiment, the proximal end of the device, the shaft and the distal end are in fluid communication (e.g., liquid and/or gas). The proximal end of the device is connected to a source of pressurized refrigerant gas or liquid. Once at the target location, the refrigerant gas or liquid is sprayed directly on the tissue through the distal end. The distal end is designed to optimize spray pattern and size or have features that facilitate the removal of exhaust gas out of the body.

In another embodiment, a principle other than the JT system is used to cool the distal end of the device to treat tissue.

In one embodiment, the cryogenic device uses nitrous oxide as the refrigerant gas. In another embodiment the cryogenic device uses carbon dioxide as the refrigerant gas. In another embodiment the cryogenic device uses any chlorofluorocarbon, hydrochlorofluorocarbon, hydrofluorocarbon or any mixtures thereof as refrigerant. In yet another embodiment the cryogenic device uses liquid nitrogen as refrigerant.

In one embodiment, the treatment is done under direct visualization. In other embodiments the treatment is performed under other forms of visualization including, but not limited, to endoscopic, bronchoscopic, fluoroscopic, sonographic, or ultrasound visualization. In other embodiments, other methods of visualization known in the art is used to assist in performing the treatment. In one embodiment a navigation tool is used to identify and facilitate access to the treatment target.

In one embodiment, the treatment procedure is performed under general anesthesia. In another embodiment, the treatment procedure is performed under local anesthesia.

According to the present invention, treatment of a target nerve, or at least a portion of the tissue it innervates, is conducted at any point along the course of the nerve or on any portion of the tissue it innervates. The treatment procedure might have to be repeated to cover the desired length of the nerve, its various anatomical locations, branches, or the desired portion of the tissue it innervates. The treatment might also need to be repeated over the course of time to maintain the therapeutic benefits of the treatment. In other embodiments, treatment might need to be repeated for more than one target nerve or tissue to achieve therapeutic benefit. In one embodiment, the target nerve, or the tissue it innervates, is accessed through the nose or the mouth. In another embodiment, the target nerve is accessed externally by creating an incision on the skin to create access.

Prior to treatment, the physician might conduct at least one diagnostic procedure to ensure of the suitability of the treatment target. In one embodiment, diagnostic step includes injecting or applying an anesthetic or another blocking agent, to the target location. In another embodiment, the diagnostic step might include examining the function of a sensory nerve using nerve stimulators, nerve visualizers or other methods known in the art to examine the function of a nerve or its receptors. In yet another embodiment, diagnosis is made by comparing the field of innervation of a given nerve with a patient's symptoms and other comorbid diseases present in the patient. As an example, cooccurrence of sleep apnea and at least one symptom of otologic dysfunction, bruxism, or nasal symptoms, can point to PhN or LPN as culprits. As another example, cooccurrence of sleep apnea, swallowing dysfunction, GI symptoms or lower respiratory symptoms can point to SLN as the main culprit. In yet another example, cooccurrence of heart failure and sleep apnea can point to SLN as the cause. In one embodiment, a diagnostic tool can be developed in form of a software, for example, using artificial intelligence, to match symptoms to innervation patterns.

During the procedure, methods known in the art can be used to visualize the target nerve. As nerves or their branches generally follow blood vessels in the airway mucosa, in one embodiment, methods known in the art to visualize arteries and blood vessels can be used to identify the approximate location of a nerve. Examples of methods used to visualize arteries include, but are not limited to, light, including visible light, magnetic resonance angiography, ultrasonography, sonography, dual red imaging, narrow band imaging, or computerized tomography.

In one method of use, upon consultation with a physician, a patient may be selected to be treated for at least one symptom of a disease according to the teachings of the current invention. Optionally, the treating physician uses a diagnostic method to identify the hyperreactive nerve or its reflex arc. Upon confirmation of the target, the physician decides whether to treat the nerve along its course, the tissue it innervates, or the nerves that participate in the nerve's reflex arcs, for example the nerves that carry parasympathetic fibers to the areas innervated by the target nerve. As the next step, the physician decides on the choice of treatment, e.g., treatment with chemical or pharmaceutical agents, treatment using surgical or mechanical means or treatment using various energy sources and devices associated with them. Treatment can be applied by the patients, e.g., in case of pharmaceutical mode of treatment, or by the physician during a procedure. The procedure can be performed intraorally, intranasally, or externally through an excision in the skin, and under an appropriate visualization. Treatment might be applied or performed at multiple sites or on multiple occasions to produce the desired therapeutic effect.

In one embodiment of method of use, a patient is diagnosed with a disease. A device with a proximal end, a distal end, and an elongated shaft in-between is used to ablate PhN as it enters the nasopharynx through the palatovaginal canal. The device is advanced through the nasal cavity under endoscopic visualization to reach PhN. Upon reaching the target, the device is used to ablate all or substantially all nerve fibers passing through the palatovaginal opening within the nasopharynx. Once the ablation procedure is complete the device is navigated out of the nasal cavity under endoscopic visualization.

In one embodiment of the method above, the disease is an otologic condition. In one example, the disease to be treated is eustachian tube dysfunction. In one embodiment of the method above, the distal end of the device comprises an effector component. In one embodiment, the effector component uses energy or cryogenic temperatures to ablate PhN.

In another embodiment of method of use, a patient is diagnosed with a disease. A device with a proximal end, a distal end, and an elongated shaft in between, is used to ablate LPN as it enters the oral cavity through the lesser palatine canal. The device is advanced through the oral cavity under visualization to reach lesser palatine foramina on the hard palate. Upon reaching the target, the device is used to ablate all or substantially all nerve fibers passing through at least one lesser palatine foramen. The treatment might be repeated to treat all lesser palatine foramina. Once the ablation procedure is complete the device is navigated out of the oral cavity under visualization.

In one embodiment of the method above, the disease is sleep disorder breathing. In one example, the disease to be treated is snoring. In one embodiment of the method above, the distal end of the device comprises an effector component. In one embodiment, the effector component uses energy or cryogenic temperatures to ablate LPN.

In another embodiment of method of use, a patient is diagnosed with a disease. A device with a proximal end, a distal end, and an elongated shaft in between, is used to ablate iSLN. To do so, an incision is made on the skin above midway between the greater horn of the hyoid bone and the thyroid cartilage to create access to iSLN as it pierces through the thyroid membrane. The device is advanced through the incision to reach and ablate iSLN under direct or ultrasonic visualization. Once the ablation procedure is complete the device is navigated out of the incision.

In one embodiment of the method above, the disease is sleep disorder breathing. In on example, the disease to be treated OSA. In another example, the disease to be treated is chronic cough. In yet another example, the disease to be treated is hypertension. In one embodiment of the method above, the distal end of the device comprises an effector component. In one embodiment, the effector component uses energy or cryogenic temperatures to ablate iSLN.

Any of the target nerves can be treated individually or in combination with other nerves. For example, in one example only PhN is treated. In another example both PhN and SLN are treated. In yet another example, only SLN is treated.

In one embodiment of the methods of use, the disease to be treated is sleep disorder breathing such as OSA, jaw movement disorders such as bruxism, hypertension, metabolic diseases such as diabetes, thyroid disease such as goiter and hypothyroidism, obesity, or heart failure and their associated symptoms and comorbidities. In another embodiment the disease is as otologic condition such as eustachian tube dysfunction, tinnitus, Meniere's disease, or hearing loss. In another embodiment the disease is lymphoid tissue hypertrophy. In yet another embodiment the disease is chronic cough.

In one embodiment of the method of use, at least one of the devices used, comprises an effector component at its distal end and uses energy to effect ablation. Examples of energy modes that can be used include, but are not limited, to radiofrequency energy, pulsed radiofrequency energy, laser, ultrasonic energy, thermal energy (either heat or extreme cold), electrical energy, electromagnetic energy, or light. In one embodiment each nerve is treated with the same or different energy modalities. In one embodiment the device is a cryogenic device. In one embodiment the distal end of the device is expandable.

In another embodiment of the method of use, at least one of the devices used is a drug delivery instrument to deliver therapeutic or chemical agents to the proximity of the target. In one embodiment the pharmaceutical or chemical agents are formulated into a sustained release formulation before being delivered. In another embodiment the drug delivery instrument is a syringe.

In another embodiment of method of use, a patient is diagnosed with a disease. A device with a proximal end, a distal end, and an elongated shaft in between is used to ablate at least a portion of tissue innervated by PhN. The device is advanced through the nasal cavity under endoscopic visualization to reach the nasopharynx. At least a portion of the distal end of the device is placed on the nasopharyngeal mucosa to ablate at least a portion of the mucosa. Upon termination of the procedure the device is navigated out of the nasal cavity.

In one embodiment of the method above, the disease is an otologic condition. In one example, the disease to be treated is eustachian tube dysfunction. In one embodiment of the method above, the distal end of the device comprises an effector component. In one embodiment, the effector component uses energy or cryogenic temperatures to ablate nasopharyngeal mucosa.

In another embodiment of method of use, a patient is diagnosed with a disease. A device with a proximal end, a distal end, and an elongated shaft in between is used to ablate at least a portion of tissue innervated by LPN. The device is advanced through the oral cavity under visualization to reach the soft palate. At least a portion of the distal end of the device is placed on the mucosa of the soft palate to ablate at least a portion of the mucosa. Upon termination of the procedure the device is navigated out of the oral cavity.

In one embodiment of the method above, the disease is sleep disorder breathing. In on example, the disease to be treated is snoring. In one embodiment of the method above, the distal end of the device comprises an effector component. In one embodiment, the effector component uses energy or cryogenic temperatures to ablate soft palate mucosa.

In another embodiment of method of use, a patient is diagnosed with a disease. A device with a proximal end, a distal end, and an elongated shaft in between is used to ablate at least a portion of tissue innervated by SLN. The device is advanced through the nasal or oral cavity under visualization to reach the oropharynx or laryngopharynx. At least a portion of the distal end of the device is placed on the mucosa of oropharyngeal or laryngopharyngeal mucosa to ablate at least a portion of the mucosa. Upon termination of the procedure the device is navigated out of the oral or nasal cavity.

In one embodiment of the method above, the disease is sleep disorder breathing. In on example, the disease to be treated OSA. In another example, the disease to be treated is chronic cough. In yet another example, the disease to be treated is hypertension. In one embodiment of the method above, the distal end of the device comprises an effector component. In one embodiment, the effector component uses energy or cryogenic temperatures to ablate a portion of oropharyngeal or laryngopharyngeal mucosa.

In one embodiment, at least one of the devices used, comprises an expandable balloon disposed at its distal end. During the procedure, the device is navigated to the target tissue, once in position the balloon is expanded using an inflation device connected to the proximal end of the device. The expanded balloon applies pressure to the target tissue to treat it. Upon completion of the procedure, the balloon is deflated and is navigated out of the body.

In another embodiment of method of use, a patient is diagnosed with a disease. A device with a proximal end, a distal end, and an elongated shaft in between, is used to compress at least a portion of the oropharyngeal or laryngopharyngeal mucosa. The device is advanced through the oral cavity under visualization to reach the target tissue. Upon reaching the target, the device is made to change configuration from a collapsed state to an expanded state to apply pressure on at least a portion of the mucosa. The treatment might be repeated to treat larger portion of the mucosa. Once treatment is complete the device is navigated out of the oral cavity under visualization. In one embodiment, the device reaches pharyngeal mucosa through the nasal cavity. In one embodiment, the treatment device is an expandable balloon.

Any of the tissue described above can be treated individually or in combination with other nerves or tissues. For example, in one example only the mucosa of the nasopharynx is treated. In another example both nasopharyngeal and pharyngeal mucosa are treated. In yet another example, only pharyngeal or laryngeal mucosa is treated.

In one embodiment the disease to be treated is sleep disorder breathing such as OSA, jaw movement disorders such as bruxism, hypertension, metabolic diseases such as diabetes, thyroid disease such as goiter and hypothyroidism, obesity, or heart failure and their associated symptoms and comorbidities. In another embodiment the disease is as otologic condition such as eustachian tube dysfunction, tinnitus, Meniere's disease, or hearing loss. In another embodiment the disease is lymphoid tissue hypertrophy. In yet another embodiment the disease is chronic cough.

In one embodiment, at least one of the devices used, comprises an effector component at its distal end and uses energy to effect ablation. Examples of energy modes that can be used include, but are not limited, to radiofrequency energy, pulsed radiofrequency energy, laser, ultrasonic energy, thermal energy (either heat or extreme cold), electrical energy, electromagnetic energy, or light. In one embodiment each nerve is treated with the same or different energy modalities. In one embodiment the device is a cryogenic device. In one embodiment the distal end of the device is expandable.

In another embodiment, at least one of the devices used is a drug delivery instrument to deliver therapeutic or chemical agents to the proximity of the target. In one embodiment the pharmaceutical or chemical agents are formulated into a sustained release formulation before being delivered.

In one embodiment of method of use, a patient is diagnosed with a disease. A device with a proximal end, a distal end, and an elongated shaft in between is used to ablate GPN as it enters the oral cavity through the greater palatine foramina. The device is advanced through the oral cavity under visualization to reach at least one greater palatine foramen. The distal end of the device is placed in the proximity of the target to treat all or substantially all fibers going through the foramen. This step can be repeated multiple times to treat all greater palatine foramina. Optionally, and if necessary, the same or another similar device is used to ablate NPN at the incisive foramen in the oral cavity. The device is advanced intraorally and under visualization to reach the incisive foramen. The distal end of the device is placed in the proximity of the incisive foramen to treat all or substantially all fibers going through the foramen. Optionally, and if necessary, the same or another similar device is used to ablate LPN at its foramina in the oral cavity. The device is advanced intraorally and under visualization to reach lesser palatine foramina. The distal end of the device is placed in the proximity of at least one of the lesser palatine foramina to treat all or substantially all fibers going through the foramen. This step can be repeated multiple times to treat all lesser foramina. In all the steps above, upon completion, the device is navigated out of the body.

Any the steps above can be performed individually or in combination with other steps. For example, in one example only GPN is treated. In another example GPN and NPN are treated. In yet another example, in addition to GPN and NPN, LPN is also treated.

In one embodiment the disease to be treated is sleep disorder breathing such as OSA, jaw movement disorder such as bruxism, temporomandibular joint disorders, palatal inflammatory diseases, or gingival or periodontal inflammatory diseases such as periodontitis, gingivitis.

In one embodiment, at least one of the devices used, comprises an effector component at its distal end and uses energy to effect ablation. Examples of energy modes that can be used include, but are not limited, to radiofrequency energy, pulsed radiofrequency energy, laser, ultrasonic energy, thermal energy (either heat or extreme cold), electrical energy, electromagnetic energy, or light. In one embodiment each nerve is treated with the same or different energy modalities. In one embodiment the device is a cryogenic device. In one embodiment the distal end of the device is expandable.

In another embodiment, at least one of the devices used is a drug delivery instrument to deliver therapeutic or chemical agents to the proximity of the target. In one embodiment the pharmaceutical or chemical agents are formulated into a sustained release formulation before being delivered. In one embodiment the drug delivery instrument is a syringe.

In another embodiment, a patient is diagnosed with bruxism. An intraoral, patient matched device, is used that covers at least a portion of the hard palate to prevent the mucosal sensory receptors to be activated upon contact with the tongue.

In another embodiment of the method of use, a patient is diagnosed with a disease. A device with a proximal end, a distal end, and an elongated shaft in between is used to ablate at least a portion of SAN, ASAN, MSAN, PSAN to treat the disease. The device is advanced through the nasal cavity under endoscopic visualization to reach the maxillary sinus. At least a portion of the distal end of the device is placed on the mucosa to ablate at least a portion of said nerves or the dental plexus. Upon termination of the procedure the device is navigated out of the nasal cavity.

In one embodiment of the method above, the disease is periodontitis. In one example, the disease to be treated is gingivitis. In one embodiment of the method above, the distal end of the device comprises an effector component. In one embodiment, the effector component uses energy or cryogenic temperatures to ablate any portion of the said nerves.

In one embodiment of the method of use, a patient is diagnosed with periodontitis. A device with a proximal end, a distal end, and an elongated shaft in between is used to ablate at least a portion of SPG to treat the disease. The device is advanced through the nasal cavity under endoscopic visualization to reach the proximity of SPG. Upon completion of the ablation procedure, the device is navigated out of the nasal cavity,

In one embodiment of the method above, the device uses energy to ablate SPG. In another embodiment a chemical or pharmaceutical agent is used to ablate SPG.

One embodiment for a method for treating dysfunctions or diseases in a patient may generally comprise positioning a treatment device having a proximal end, a distal end, an elongate shaft therebetween, and an ablation effector disposed at or near the distal end of the device into proximity of a superior laryngeal nerve or any of its branches, and ablating at least a portion of the superior laryngeal nerve or any of its branches thereof using the ablation effector until at least one symptom of nerve dysfunction or disease is reduced.

In another aspect of the method, the dysfunctions or diseases to be treated is selected from the group consisting of sleep disorder breathing, sleep apnea, obstructive sleep apnea, snoring, central sleep apnea, heart failure, cardiovascular disease, hypertension, temporomandibular joint disorder (TMD), renal disease, pulmonary disease, diabetes, obesity, dyslipidemia, thyroid disease, such as goiter and hypothyroidism, obesity, neuropsychiatric diseases, metabolic disease, neuroimmune disease, dysphagia, swallowing disorders, gastrointestinal disorders, lower respiratory tract disorders, cough, chronic cough, voice disturbances, otologic conditions such as eustachian tube dysfunction, tinnitus, ear fullness, Meniere's disease, hearing loss, bruxism, gingival and periodontal inflammatory conditions, mouth breathing, anxiety, depression, daytime sleepiness, wheezing, nasopharyngeal symptoms such as postnasal drip, water in the throat, throat dryness, rhinorrhea, nasal obstruction, lymphoid tissue hypertrophy, laryngeal hyperreactivity syndrome, or other disorders that are directly or indirectly associated with hyperreactivity of the aforementioned nerve.

In another aspect of the method, ablating at least the portion of the superior laryngeal nerve comprises ablating an internal branch of the superior laryngeal nerve.

In another aspect of the method, positioning the treatment device comprises transcutaneously accessing the internal branch of the superior laryngeal nerve.

In another aspect of the method, ablating at least the portion of the superior laryngeal nerve comprises ablating at least a portion of oropharyngeal or laryngopharyngeal mucosa.

In another aspect of the method, ablating at least the portion of oropharyngeal or laryngopharyngeal mucosa comprises applying pressure or mechanical force on the mucosa via the treatment device.

In another aspect of the method, applying pressure or mechanical force on the mucosa comprises applying the pressure or mechanical force via an expandable balloon catheter.

In another aspect of the method, ablating at least the portion of the superior laryngeal nerve comprises cryo-ablating via the treatment device.

In another aspect of the method, ablating at least the portion of the superior laryngeal nerve comprises applying energy to the portion via the treatment device.

In another aspect of the method, the energy comprises bipolar radiofrequency, pulsed radiofrequency, cooled radiofrequency, microwave, laser, ultrasonic energy, thermal energy, or combinations thereof.

In another aspect of the method, ablating at least the portion of the superior laryngeal nerve comprises applying a chemical agent to the portion via the treatment device.

In another aspect of the method, the chemical agent comprises glycerol, ethanol, phenol, or combination thereof.

In another aspect of the method, ablating at least the portion of the superior laryngeal nerve comprises applying a therapeutic agent to the portion via the treatment device.

In another aspect of the method, therapeutics agent comprises TRP channel inhibitors, TRPA1 and TRPV1 antagonist, substance P antagonist, CGRP antagonist, P2×3 antagonist, P2×2 antagonists, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin, neurotoxin agents, anesthetics, analgesics, corticosteroids, VIP antagonist, muscarinic antagonists, nicotinic antagonists, anticholinergic agents, or combinations thereof, beta blockers, beta-2 (β2) agonists and alpha-2 (α2) agonists.

In another aspect of the method, the method may further comprise visualizing at least the portion of the superior laryngeal nerve while ablating.

In another aspect of the method, the method may further comprise illuminating at least the portion of the superior laryngeal nerve while ablating.

Yet another embodiment for a method for treating dysfunctions or diseases in a patient may generally comprise introducing a treatment device into a nasopharynx of the patient, the treatment device having a proximal end, a distal end, an elongate shaft therebetween, and an ablation effector disposed at or near the distal end of the device, advancing the distal end of the device under visualization into proximity of a palatovaginal canal (PVC) opening within the nasopharynx, and ablating all or substantially all of the pharyngeal nerve fibers passing through the PVC opening using the ablation effector until at least one symptom of nerve dysfunction or disease is reduced.

In another aspect of the method, the dysfunctions or diseases to be treated is selected from the group consisting of sleep disorder breathing, sleep apnea, obstructive sleep apnea, snoring, central sleep apnea, heart failure, cardiovascular disease, hypertension, temporomandibular joint disorder (TMD), renal disease, pulmonary disease, diabetes, obesity, dyslipidemia, thyroid disease, such as goiter and hypothyroidism, obesity, neuropsychiatric diseases, metabolic disease, neuroimmune disease, dysphagia, swallowing disorders, gastrointestinal disorders, lower respiratory tract disorders, cough, chronic cough, voice disturbances, otologic conditions such as eustachian tube dysfunction, tinnitus, ear fullness, Meniere's disease, hearing loss, otitis media, chronic otitis media, otitis media with effusion, chronic suppurative otitis media, bruxism, gingival and periodontal inflammatory conditions, mouth breathing, anxiety, depression, daytime sleepiness, wheezing, nasopharyngeal symptoms such as postnasal drip, water in the throat, throat dryness, rhinorrhea, nasal obstruction, lymphoid tissue hypertrophy, laryngeal hyperreactivity syndrome, or other disorders that are directly or indirectly associated with hyperreactivity of the aforementioned nerve.

In another aspect of the method, ablating all or substantially all of the pharyngeal nerve fibers comprises applying pressure or mechanical force on a mucosa covering the PVC opening within the nasopharynx.

In another aspect of the method, applying pressure or mechanical force comprises applying the pressure or mechanical force via an expandable balloon catheter.

In another aspect of the method, ablating all or substantially all of the pharyngeal nerve fibers comprises cryo-ablating via the treatment device.

In another aspect of the method, ablating all or substantially all of the pharyngeal nerve fibers comprises applying energy to the pharyngeal nerve fibers via the treatment device.

In another aspect of the method, the energy comprises bipolar radiofrequency, pulsed radiofrequency, cooled radiofrequency, microwave, laser, ultrasonic energy, thermal energy, or combinations thereof.

In another aspect of the method, ablating all or substantially all of the pharyngeal nerve fibers comprises applying a chemical agent to the pharyngeal nerve fibers.

In another aspect of the method, the chemical agent comprises glycerol, ethanol, phenol, or combination thereof.

In another aspect of the method, ablating all or substantially all of the pharyngeal nerve fibers comprises applying a therapeutic agent to the pharyngeal nerve fibers.

In another aspect of the method, therapeutics agent comprises TRP channel inhibitors, TRPA1 and TRPV1 antagonist, substance P antagonist, CGRP antagonist, P2×3 antagonist, P2×2 antagonists, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin, neurotoxin agents, anesthetics, analgesics, corticosteroids, VIP antagonist, muscarinic antagonists, nicotinic antagonists, anticholinergic agents, or combinations thereof, beta blockers, beta-2 (β2) agonists and alpha-2 (α2) agonists.

In another aspect of the method, the method may further comprise visualizing the pharyngeal nerve fibers while ablating.

In another aspect of the method, the method may further comprise illuminating the pharyngeal nerve fibers while ablating.

Yet another embodiment of a method for treating dysfunctions or diseases in a patient may generally comprise introducing a treatment device into the oral or nasal cavity of the patient, the treatment device having a proximal end, a distal end, an elongate shaft therebetween, and an ablation effector disposed at or near the distal end of the device, ablating all or substantially all of the lesser palatine nerve fibers using the ablation effector until at least one symptom of nerve dysfunction or disease is reduced.

In another aspect of the method, the dysfunctions or diseases to be treated is selected from the group consisting of sleep disorder breathing, sleep apnea, obstructive sleep apnea, snoring, central sleep apnea, heart failure, cardiovascular disease, hypertension, temporomandibular joint disorder (TMD), renal disease, pulmonary disease, diabetes, obesity, dyslipidemia, thyroid disease, such as goiter and hypothyroidism, obesity, neuropsychiatric diseases, metabolic disease, neuroimmune disease, dysphagia, swallowing disorders, gastrointestinal disorders, lower respiratory tract disorders, cough, chronic cough, voice disturbances, otologic conditions such as eustachian tube dysfunction, tinnitus, ear fullness, Meniere's disease, hearing loss, bruxism, gingival and periodontal inflammatory conditions, mouth breathing, anxiety, depression, daytime sleepiness, wheezing, nasopharyngeal symptoms such as postnasal drip, water in the throat, throat dryness, rhinorrhea, nasal obstruction, lymphoid tissue hypertrophy, laryngeal hyperreactivity syndrome, or other disorders that are directly or indirectly associated with hyperreactivity of the aforementioned nerve.

In another aspect of the method, introducing the treatment device comprises introducing through the nasal cavity.

In another aspect of the method, introducing the treatment device through the nasal cavity comprises advancing the distal end of the treatment device into the maxillary sinus of the patient to the proximity of lesser palatine canal.

In another aspect of the method, introducing the treatment device comprises introducing through the oral cavity.

In another aspect of the method, introducing the treatment device through the oral cavity comprises advancing the distal end of the treatment device into the proximity of at least one lesser palatine foramen.

In another aspect of the method, ablating all or substantially all of the lesser palatine fibers comprises ablating at least a portion of a soft palate mucosa.

In another aspect of the method, ablating all or substantially all of the lesser palatine fibers comprises ablating at least a portion of levator veli palatini, palatopharyngeus, or musculus uvula.

In another aspect of the method, ablating all or substantially all of the lesser palatine fibers comprises cryo-ablating via the treatment device.

In another aspect of the method, ablating all or substantially all of the lesser palatine fibers comprises applying energy to the lesser palatine fibers.

In another aspect of the method, the energy comprises bipolar radiofrequency, pulsed radiofrequency, cooled radiofrequency, microwave, laser, ultrasonic energy, thermal energy, or combinations thereof.

In another aspect of the method, ablating all or substantially all of the lesser palatine fibers comprises applying a chemical agent to the lesser palatine fibers.

In another aspect of the method, the chemical agent comprises glycerol, ethanol, phenol, or combination thereof.

In another aspect of the method, ablating all or substantially all of the lesser palatine fibers comprises applying a therapeutic agent to the lesser palatine fibers.

In another aspect of the method, therapeutics agent comprises TRP channel inhibitors, TRPA1 and TRPV1 antagonist, substance P antagonist, CGRP antagonist, P2×3 antagonist, P2×2 antagonists, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin, neurotoxin agents, anesthetics, analgesics, corticosteroids, VIP antagonist, muscarinic antagonists, nicotinic antagonists, anticholinergic agents, or combinations thereof. beta blockers, beta-2 (β2) agonists and alpha-2 (α2) agonists.

In another aspect of the method, the method may further comprise visualizing the lesser palatine fibers while ablating.

Yet another embodiment for a method for treating sleep disorder breathing in a patient may generally comprise positioning a treatment device having a proximal end, a distal end, an elongate shaft therebetween, and an ablation effector disposed at or near the distal end of the device into proximity of a superior laryngeal nerve, and ablating at least a portion of the superior laryngeal nerve using the ablation effector until at least one symptom of sleep disorder breathing is reduced.

Yet another embodiment for a method for treating sleep apnea in a patient may generally comprise positioning a treatment device having a proximal end, a distal end, an elongate shaft therebetween, and an ablation effector disposed at or near the distal end of the device into proximity of a superior laryngeal nerve, and ablating at least a portion of superior laryngeal nerve using the ablation effector until at least one symptom of sleep apnea is reduced.

In another aspect of the method, sleep apnea comprises obstructive sleep apnea or central sleep apnea.

Yet another embodiment for a method for treating chronic cough in a patient may generally comprise positioning a treatment device having a proximal end, a distal end, an elongate shaft therebetween, and an ablation effector disposed at or near the distal end of the device into proximity of a superior laryngeal nerve, and ablating at least a portion of superior laryngeal nerve using the ablation effector until at least one symptom of sleep apnea is reduced.

Yet another embodiment for a method for treating heart failure in a patient may generally comprise positioning a treatment device having a proximal end, a distal end, an elongate shaft therebetween, and an ablation effector disposed at or near the distal end of the device into proximity of a superior laryngeal nerve, and ablating at least a portion of superior laryngeal nerve using the ablation effector until at least one symptom of heart failure is reduced.

Yet another embodiment for a method for treating hypertension in a patient may generally comprise positioning a treatment device having a proximal end, a distal end, an elongate shaft therebetween, and an ablation effector disposed at or near the distal end of the device into proximity of a superior laryngeal nerve, and ablating at least a portion of superior laryngeal nerve using the ablation effector until at least one symptom of hypertension is reduced.

Yet another embodiment for a method for treating diabetes in a patient may generally comprise positioning a treatment device having a proximal end, a distal end, an elongate shaft therebetween, and an ablation effector disposed at or near the distal end of the device into proximity of a superior laryngeal nerve, and ablating at least a portion of superior laryngeal nerve using the ablation effector until at least one symptom of diabetes is reduced.

Yet another embodiment for a method for treating obesity in a patient may generally comprise positioning a treatment device having a proximal end, a distal end, an elongate shaft therebetween, and an ablation effector disposed at or near the distal end of the device into proximity of a superior laryngeal nerve, and ablating at least a portion of superior laryngeal nerve using the ablation effector until at least one symptom of obesity is reduced.

Yet another embodiment for a method for treating sleep disordered breathing in a patient may generally comprise positioning a treatment device having a proximal end, a distal end, an elongate shaft therebetween, and an ablation effector disposed at or near the distal end of the device into proximity of a lesser palatine nerve, and ablating at least a portion of lesser palatine nerve using the ablation effector until at least one symptom of sleep disordered breathing is reduced.

Yet another embodiment for a method for treating sleep apnea in a patient may generally comprise positioning a treatment device having a proximal end, a distal end, an elongate shaft therebetween, and an ablation effector disposed at or near the distal end of the device into proximity of a lesser palatine nerve, and ablating at least a portion of lesser palatine nerve using the ablation effector until at least one symptom of sleep apnea is reduced.

Yet another embodiment for a method for treating snoring in a patient may generally comprise positioning a treatment device having a proximal end, a distal end, an elongate shaft therebetween, and an ablation effector disposed at or near the distal end of the device into proximity of a lesser palatine nerve, and ablating at least a portion of lesser palatine nerve using the ablation effector until at least one symptom of snoring is reduced.

Yet another embodiment for a method for treating sleep disordered breathing in a patient may generally comprise positioning a treatment device having a proximal end, a distal end, an elongate shaft therebetween, and an ablation effector disposed at or near the distal end of the device into proximity of a pharyngeal nerve, and ablating at least a portion of pharyngeal nerve using the ablation effector until at least one symptom of sleep disordered breathing is reduced.

Yet another embodiment for a method for treating otologic conditions in a patient may generally comprise positioning a treatment device having a proximal end, a distal end, an elongate shaft therebetween, and an ablation effector disposed at or near the distal end of the device into proximity of a pharyngeal nerve, and ablating at least a portion of pharyngeal nerve using the ablation effector until at least one symptom of otologic conditions is reduced.

Yet another embodiment for a method for treating eustachian tube dysfunction in a patient may generally comprise positioning a treatment device having a proximal end, a distal end, an elongate shaft therebetween, and an ablation effector disposed at or near the distal end of the device into proximity of a pharyngeal nerve, and ablating at least a portion of pharyngeal nerve using the ablation effector until at least one symptom of eustachian tube dysfunction is reduced.

Yet another embodiment for a method for otitis media in a patient may generally comprise positioning a treatment device having a proximal end, a distal end, an elongate shaft therebetween, and an ablation effector disposed at or near the distal end of the device into proximity of a pharyngeal nerve, and ablating at least a portion of pharyngeal nerve using the ablation effector until at least one symptom of otitis media is reduced.

### BRIEF DESCRIPTION OF THE DRAWINGS

FIG. 1 shows the schematic overview of the upper airway and the sensory, muscular, and mucosal components that govern airway patency.

FIG. 2A shows the schematic sagittal cross sections of the nasal cavity and the roof of the mouth, showing nasal cavity 200, soft palate 201, and the nasopharynx 202.

FIG. 2B shows the schematic of a traverse plane of the roof of the mouth, illustrating the hard palate 213 and soft palate 201.

FIG. 2C shows the greater petrosal nerve 217 and maxillary nerve 218 merge within sphenopalatine ganglion 203, to provide sensory, parasympathetic, and sympathetic fibers to pharyngeal nerve 206, lesser palatine nerve 204, greater palatine nerve 212, and nasopalatine nerve 210.

FIG. 3A shows the schematic of the upper larynx, illustrating the internal branch of the superior laryngeal nerve 301, piercing through the thyroid membrane 303 to provide sensory innervation to the laryngeal mucosa.

FIG. 3B shows the approximate location of the area on the neck above the point where laryngeal nerve 301 pierces through the thyroid membrane 303.

FIG. 3C shows the side view of the head and neck illustrating the innervation of the oropharynx by pharyngeal branch of the glossopharyngeal nerve 313 and the pharyngeal branch of the vagus nerve 314.

FIG. 4 shows the sagittal cross section of the head and neck, pointing to the approximate area of tissues innervated by pharyngeal and lesser palatine nerves 401, the approximate area of tissues innervated by superior laryngeal nerve 402, and the approximate area of tissues innervated by pharyngeal branches of glossopharyngeal and vagus nerves 403.

FIG. 5A shows the schematic side views of one example of a device according to this invention, having a proximal end 501, distal end 502, and an elongated shaft in between. Distal end 502 can assume a collapsed configuration or can be expanded to assume expanded configuration 502′.

FIG. 5B shows the sagittal cross section of the head with nasal cavity 504 and the oral cavity 505. The figure shows one configuration of treatment device 500 where the device 500 is an expandable balloon. The device is shown having reached the nasopharynx 506 through the nasal cavity and been expanded to acquire expanded configuration 502′ in order to apply pressure on nasopharyngeal mucosa.

FIG. 5C shows the sagittal cross section of the head with oral cavity 505. The figure shows one configuration of treatment device 500 where the device 500 is an expandable balloon. The device is shown having reached the pharynx 507 through the oral cavity and been expanded to acquire expanded configuration 502′ to apply pressure on the oropharyngeal or laryngopharyngeal mucosa.

FIG. 5D shows the sagittal cross section of the head with nasal cavity 504. The figure shows one configuration of treatment device 500 where the device 500 is an energy-based device. The distal end of device is shown having reached the nasopharynx 506 through the nasal cavity to treat the nasopharyngeal mucosa or the PhN fibers going through the palatovaginal canal opening 508 in the nasopharynx.

FIG. 5E shows the schematic of a person lying on supine position with their mouth open. The tissue covering the oral cavity is made transparent to allow for viewing of the airway. The figure shows one configuration of treatment device 500 where the device 500 is an energy-based device. The distal end of device is shown having reached the pharynx 507 through the oral cavity to treat the oropharyngeal or laryngopharyngeal mucosa.

FIG. 6A shows the roof of the mouth with hard palate 601 and soft palate 602.

FIG. 6B shows a portion of hard palate 601 being covered by physical barrier 603.

FIG. 7 shows the schematic of the upper and lower jaw and teeth and the nerves that provide sensory innervation to the maxillary and mandibular gum and gingivae.

FIG. 8 shows a flow chart of an example of a method to treat diseases where a target nerve is treated.

FIG. 9 shows a flow chart of an example of a method to treat diseases where the tissue innervated by a target nerve is treated.

### DETAILED DESCRIPTION OF THE INVENTION

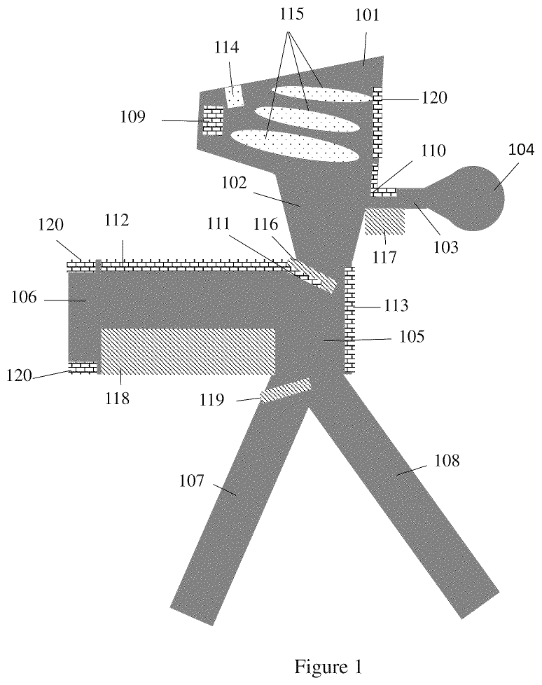

FIG. 1 shows a schematic of the upper airway comprising nasal cavity 101, nasopharynx 102, auditory tube 103, middle ear 104, pharynx (including both oropharynx and laryngopharynx) 105, oral cavity 106, larynx 107 and esophagus 108. The upper airway is constantly monitored by an array of sensors innervated by the trigeminal, glossopharyngeal, and the vagus nerves. These sensory neurons, monitor the environment and potentiate a response in accordance with the input. The responses are in form of local release of neuropeptides to activate immune cells or act on blood vessels, or are centrally mediated motor responses to upper airway muscles, or are in form of centrally mediated parasympathetic reflexes to induce mucus secretion and vasodialation. The outcome of this coordinated sensing-response feedback loop is the adjustment of upper airway resistance, patency, and coordination of functions such as breathing, eating, drinking, or speaking. FIG. 1 depicts upper airway sensors, shown as brick pattered rectangles, that include nasal cavity sensors positioned behind the nasal vestibule, designated as 109 and innervated by anterior ethmoid nerve, AEN, not shown. The posterior nasal nerves (PNN) provide sensory innervation to the mucosal lining of the posterior aspect of the nasal cavity and the turbinates. These sensors are schematically depicted and are designated 120. The sensors in the nasopharynx and those within the auditory tube are designated 110 and are innervated by pharyngeal branch of maxillary nerve (PhN), also not shown. Sensory innervation of the soft palate and the inferior aspect of the nasopharynx are designated as 111 and are provided by lesser palatine nerve (LPN), not shown. Sensory neurons of the palate, are designated as 112 and are provided by greater palatine and nasopalatine nerves. Pharyngeal branch of the glossopharyngeal nerve (PhIX), pharyngeal branch of the vagus nerve (PhX), and superior laryngeal nerve (SLN), provide sensory innervation to the oropharynx and laryngopharynx designated 113. Sensory innervation to the gingival and periodontal tissues, designated 120, is provided by the superior and inferior alveolar nerves.

The motor response resulting from sensory neuron signaling activates upper airway muscles to open or close the airway or facilitate functions such as mastication, swallowing, and breathing. Some of upper airway muscles are shown in form of rectangles with diagonal line patterns in FIG. 1 and include soft palate 116, tensor veli palatini muscle 117, the tongue 118, and epiglottis 119.

In addition to motor response, the parasympathetic reflex initiated by the sensory input, acts on arteries and veins embedded within the mucosa, affecting mucosal thickness, which also impacts airway resistance and patency. The autonomic response can also activate secretory cells to secrete mucus to wash out an offending agent or lubricate the airway. Of note, the nasal valve 114 and nasal turbinates 115, can increase in size due to the action of the parasympathetic reflex to modify airway resistance.

In some instances, the parasympathetic and sympathetic fibers that innervate the mucosa of the airway reach their target tissue by merging with the same nerve that carries the sensory fibers. This is the case for tissues innervated by PhN, GPN, NPN, PhIX, PhX and SLN. In other cases, the parasympathetic and sympathetic innervation (together referred to as autonomic innervation) is provided by a different nerve.

A malfunctioning sensory nerve can impact the movement of upper airway muscles (opening or closing the airway), mucosal thickness, airway patency or mucus secretion. These occur either due to the direct impact of the sensory nerve on the mucosa or blood vessels or is potentiated through a reflex arc that is mediated by central connections of the sensory neurons. Therefore, to treat a malfunction, one possible pathway is to disconnect the malfunctioning nerve itself. Another approach is to block the reflex arcs that the malfunctioning nerve employs to exert its influence.

The activity of sensory neurons has as a significant impact on the function and patency of the upper airway and their dysfunction can result in conditions such as sleep apnea, rhinitis, bruxism, ear pathologies, jaw movement pathologies, temporomandibular joint disorder, and gum disease such as gingivitis and periodontitis. The current disclosure teaches methods to treat the malfunctioning sensory neurons, by either treating the sensory nerves, the receptors associated with the sensory nerve that are present in the tissue they innervate, the nerve that provides the autonomic innervation to the tissues the malfunctioning sensory innervates, or the autonomic nerve fibers or effector cells that are resident in the tissue and are activated by the sensory neuron through a reflex arc. The choice of the target to be treated is determined by the disease, its symptoms, and the pattern by which they present in a patient. Matching the location where the symptoms occur to a sensory nerve's pattern of innervation and its motor and autonomic reflex arcs can help identify the tissue or the nerve(s) to be treated. The mode of treatment also depends on the accessibility of the nerve and tissues. For example, some nerves are accessible through non-invasive methods while others are not. Treating the tissue instead of disconnecting the nerve requires a larger surface area to be treated but provides an opportunity to treat both the sensory and autonomic pathways at the same time.

Embodiments provided in this disclosure are examples of treatments that can be performed to affect the sensory-feedback loops that govern the upper airway function and are by no means exhaustive. Other variations of targets to treat, diseases to treat and modes of treatment can be implied from the map presented in disclosure as FIG. 1.

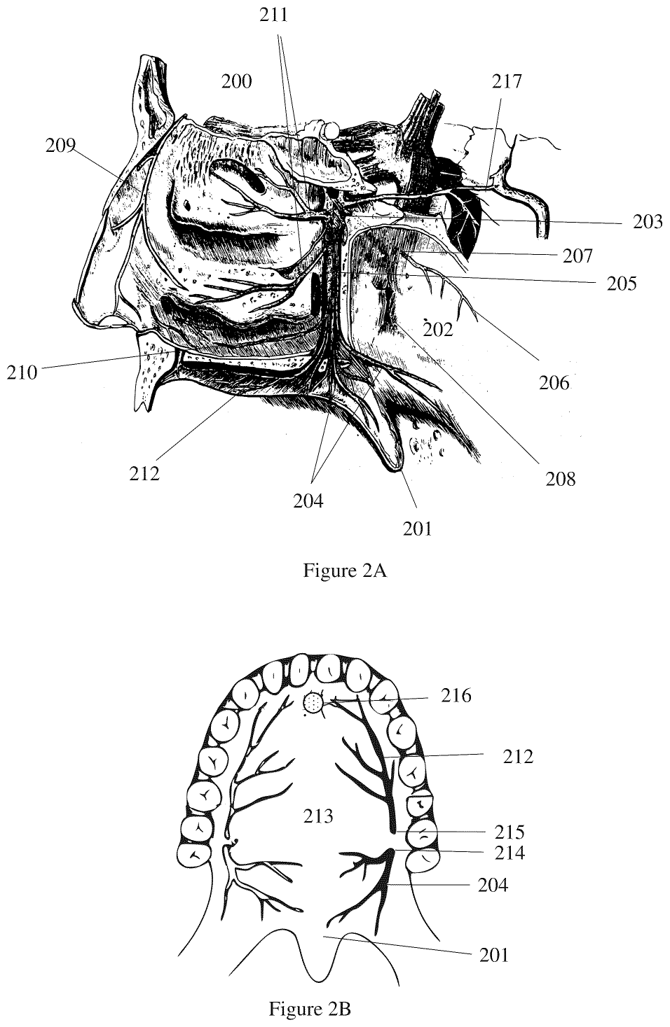

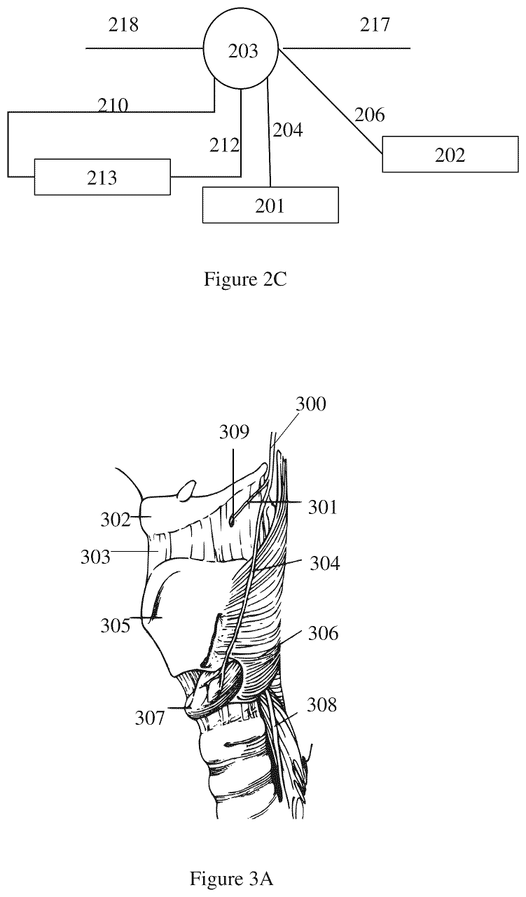

FIG. 2A shows the sagittal view of the nasal cavity 200, soft palate 201, nasopharynx 202, sphenopalatine ganglion (SPG) 203, lesser palatine nerve (LPN) 204. The LPN originates from SPG 203 and courses through the palatine canal(s) 205 (shown as an opening into the bony cavity) and emerges on the oral side of the palate through the lesser palatine foramen (LPF), not shown in this figure. Pharyngeal nerve (PhN) 206 originates from SPG 203, courses through palatovaginal canal (not shown), and emerges in the nasopharynx 202 through the palatovaginal foramen (PVF) 207 in the proximity of tubal orifice (eustachian tube opening into the nasopharynx) 208. FIG. 2A also depicts anterior ethmoid nerve (AEN) 209, nasopalatine nerve (NPN) 210, posterior nasal nerves (PNNs) 211, and greater palatine nerve (GPN) 212. GPN 212 leaves SPG 203, coursing through palatine canal(s) 205, and emerges on the oral surface of the hard palate though the greater palatine foramina (GPF), not shown in this figure. LPN 204 and GPN 212 travel through the same or two separate bony canals from SPG 203 to emerge on the oral side of the palate. The specifics of bony canals are not depicted in FIG. 1A for clarity purposes. The greater petrosal nerve (GPeN) 217 brings both sympathetic and parasympathetic fibers from the facial nerve and internal carotid plexus to the maxillary and infraorbital nerves (not shown) and SPG. These autonomic fibers are then distributed to PhN, LPN, NPN, GPN and other branches of the maxillary nerve.

FIG. 2B shows the traverse view of the hard palate 213 and the soft palate 201. The LPN 204 emerges on the surface of the palate 213 through lesser palatine foramen (LPF) 214 and courses posteriorly toward soft palate 201 The GPN emerges on the palate 213 through greater palatine foramen (GPF) 215 and courses anteriorly on the hard palate 213. In some anatomical variations, not shown here, LPN 204 and GPN 212, arrive on the palate 213 through the same foramen. In other variations, not shown here, GPN 212 and LPN 204 emerge on the hard palate 213 through multiple foramina. Nasopalatine nerve 210, emerges on the palate 213, through the incisive foramen (IF) 216.

FIG. 2C, shows the schematic of the nerves that innervate the nasopharynx 202, the soft 201 and hard palate 213. Sensory innervation from the maxillary branch of the trigeminal nerve (V2) 218, is mixed with parasympathetic and sympathetic fibers carried by the greater petrosal nerve (GPeN) 217 in SPG 203. The PhN 206, LPN 204, NPN 210, and GPN 212, carry the mixed sensory, parasympathetic, and sympathetic fibers to nasopharynx 202, soft palate 201, and hard palate 213, respectively.

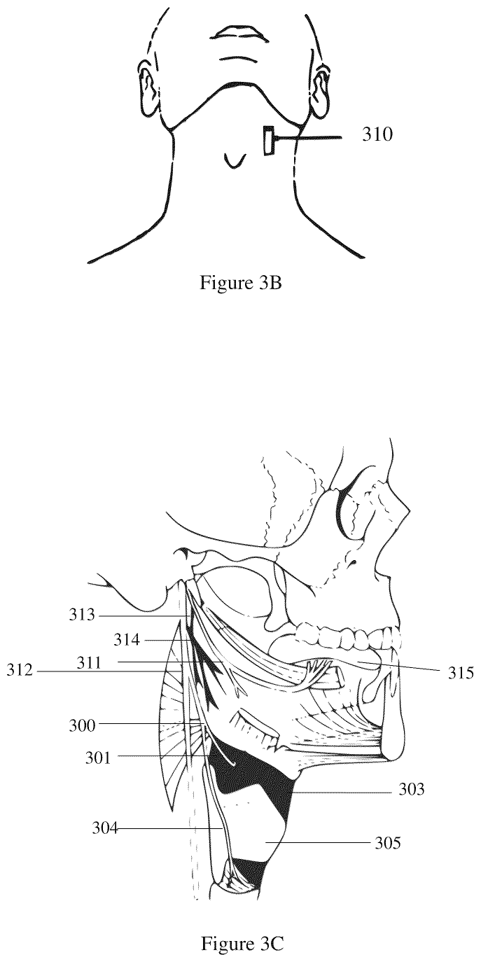

FIG. 3A depicts the side view of the larynx with superior laryngeal nerve (SLN) 300, internal branch of superior laryngeal nerve (iSLN) 301, hyoid bone 302, thyroid membrane 303, external branch of superior laryngeal nerve (eSLN) 304, thyroid cartilage 305, cricothyroid muscle 306, cricoid cartilage 307, recurrent laryngeal nerve (RLN) 308. Area 309 points to the location where iSLN 301 pierces through thyroid membrane 303 to provide sensory innervation to the mucosa of the larynx.

FIG. 3B shows frontal view of a head and neck of a patient. Area 310, points to an area on the patient's neck that is located approximately above point 309, as depicted in FIG. 3A, where iSLN 301 pierces through thyroid membrane 303. An incision or tissue puncture within area 310 can allow access to iSLN 301, providing a non-invasive route to treat iSLN 301.

FIG. 3C shows a drawing of the head and neck to show nerves that innervate the mucosa of the oropharynx and laryngopharynx. Glossopharyngeal nerve 311 innervates posterior aspect of the tongue 315. Pharyngeal branch of the glossopharyngeal nerve (PhIX) 313 (highlighted in black) innervates the mucosa of the oropharynx. The vagus nerve 312 innervates the oropharynx mucosa through its pharyngeal branch (PhX) 314 (also highlighted in black) and then branches into SLN 300, iSLN 301, and external branch of SLN 304. PhIX, PhX and iSLN form a dense, interconnected network to innervate the mucosa of the oropharynx and laryngopharynx.

### Sleep Disorder Breathing, Heart Failure, Otologic Conditions

In one embodiment, at least one of PhN 206, LPN 204, PhIX 313, PhX 314, SLN 300 and iSLN 301, or at least a portion of the tissue they innervate, is treated to treat or reduce symptoms associated with sleep disorder breathing disorders (SBD), a class of disorders that include but not limited to upper airway resistance syndrome, central apnea, obstructive sleep apnea (OSA), mixed apnea, or snoring. In one embodiment, treating the tissue innervated by at least one of PhN 206, LPN 204, PhIX 313, PhX 314, SLN 300 and iSLN 301 to treat SBD include but is not limited to treating at least a portion of one of nasopharyngeal, oropharyngeal, or laryngopharyngeal mucosa.

In one embodiment, at least a portion of a PhN 206, LPN 204, PhIX 313, PhX 314, SLN 300 and iSLN 301, or at least a portion of the tissue they innervate, is treated in order to address symptoms associated with sleep disorders, sleep disorder breathing disease (SBD), heart failure, cardiovascular disease, hypertension, temporomandibular joint disorder (TMD), renal disease, pulmonary disease, diabetes, neuropsychiatric diseases, metabolic disease, neuroimmune disease, dysphagia, gastrointestinal disorders, lower respiratory tract disorders, cough, voice disturbances, otologic conditions such as eustachian tube dysfunction, tinnitus, ear fullness, Meniere's disease, hearing loss, jaw movement disorders, bruxism, gingival and periodontal inflammatory conditions, mouth breathing, anxiety, depression, daytime sleepiness, wheezing, nasopharyngeal symptoms such as postnasal drip, water in the throat, throat dryness, chronic cough, rhinorrhea, nasal obstruction, lymphoid tissue hypertrophy, laryngeal hyperreactivity syndrome, or other disorders that are directly or indirectly associated with hyperreactivity of the aforementioned nerves. In one embodiment, treating the tissue innervated by at least one of PhN 206, LPN 204, PhIX 313, PhX 314, SLN 300 and iSLN 301 to treat a disease include but is not limited to treating at least a portion of one of nasopharyngeal, oropharyngeal, or laryngopharyngeal mucosa.

In another embodiment, at least one of a PhN 206, LPN 204, PhIX 313, PhX 314, SLN 300 and iSLN 301, or at least a portion of the tissue they innervate, is treated to treat heart failure or symptoms associated with it, including, and not limited, to bradycardia, arrhythmia, SBD, hypertension, shortness of breath, fatigue, and weakness, swelling in the legs, ankles and feet, rapid or irregular heartbeat, wheezing, cough, fluid buildup, nausea and lack of appetite, difficulty concentrating or decreased alertness. In one embodiment, treating the tissue innervated by at least one of PhN 206, LPN 204, PhIX 313, PhX 314, SLN 300 and iSLN 301 to treat heart failure include but is not limited to treating at least a portion of one of nasopharyngeal, oropharyngeal, or laryngopharyngeal mucosa.

In one embodiment, at least one of PhN 206, and LPN 204, or at least a portion of the tissue they innervate, is treated to treat, or reduce symptoms of otologic conditions such as, but not limited to, eustachian tube dysfunction, tinnitus, ear fullness, Meniere's disease, and hearing loss. In one embodiment treating at least one of PhN 206, and LPN 204, to treat ontological conditions, comprises treating at least a portion of nasopharyngeal mucosa.

In one embodiment, at least one of PhN 206, and LPN 204, or at least a portion of the tissue they innervate, is treated to treat, or reduce symptoms of nasopharyngeal symptoms such as mucosal inflammation, coughing, sneezing, postnasal drip, water in the throat, throat dryness, rhinorrhea, lymphoid tissue inflammation such as adenoid hypertrophy, or tonsillar hypertrophy. In one embodiment treating at least one of PhN 206, and LPN 204, to treat nasopharyngeal symptoms, comprises treating at least a portion of nasopharyngeal mucosa.

In one embodiment, treating a nerve comprises of methods to modify the activity of the said nerve. Example of such strategies include but are not limited to; method to silence, block, ablate, resect, reduce the activity, reduce the frequency of firing, reduce the amplitude of firing, modify the threshold of firing, modify the receptors, block the receptors, modify the threshold of the receptors, or in general reduce the hyperreactivity of a nerve. In one embodiment, treating a nerve comprises treating the nerve itself, its branches, or ganglia. In another embodiment, treating a nerve comprises treating at least a portion of the tissue the nerve or its branches innervate.

In another embodiment, treating the tissue a nerve innervates include treating at least a portion of sensory, parasympathetic, or sympathetic fibers that are embedded within the tissue. In yet another embodiment, treating the tissue a nerve innervates, comprises treating at least a portion of the resident cells within the tissue. Examples of resident cells include, but are not limited to, secretory cells, goblet cells, basal cells, epithelial cells, glands, ciliary cells, solitary chemosensory cells, lymphocytes, or lymphoid follicle. In one embodiment, treating the tissue a nerve innervates comprises treating at least one blood vessel running within that tissue.

In one embodiment, treating LPN 204 comprises treating it anywhere along its course, ganglia or branches. In one embodiment LPN 204 is treated at its foramina, LPF 214, as it enters the oral cavity. In another embodiment LPN is treated on its course through the bony canal 205 from SPG 203 to LPF 214. In another embodiment LPN 204 is treated along its course on or in the soft palate 201. In one embodiment, LPN 204 is treated intranasally. In another embodiment, LPN 204 is treated intraorally. In one embodiment, more than one LPF is treated.

In one embodiment, treating PhN 206 comprises treating it anywhere along its course, ganglia, or branches. In another embodiment, PhN 106 is treated at its foramina, PVF 207, as it enters the nasopharynx. In another embodiment PhN 206 is treated on its course through the sphenoid sinus (not shown), the palatovaginal canal (not shown), or along its course from SPG 203 to PVF 207. In another embodiment PhN 206 is treated along its course in nasopharynx 202. In one embodiment, PhN 206 is treated intranasally. In another embodiment, PhN 206 is treated intraorally.

In one embodiment, treating iSLN 301 comprises treating it anywhere along its course, ganglia, or branches. In one embodiment, iSLN 301 is treated at or around area designated as 309, in FIG. 3B, where it pierces the thyroid membrane. In another embodiment, iSLN 301 is treated by creating an access through the skin in the approximate area of 310 as shown in FIG. 3B.

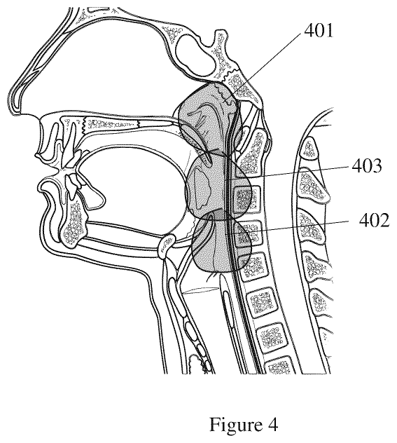

FIG. 4 shows the sagittal cross section of the head and neck. The darkened circles 401 show the approximate areas of tissues innervated by LPN 204 and PhN 206. The darkened circle 402, shows the approximate location of tissues innervated by iSLN 301. The darkened circle 403, shows the approximate location of tissues innervated by PhIX 313, PhX 314, and iSLN 301.

In one embodiment, treating LPN 204 include treating at least a portion of mucosa of nasopharynx or the soft palate, as shown approximately in 401. In another embodiment treating PhN 206 including treating at least a portion of nasopharyngeal mucosa as shown in FIG. 4 as area 401. In another embodiment, treating PhIX 313, PhX 314, SLN 300, or iSLN 301 includes treating at least a portion of oropharyngeal mucosa, for example, treating posterior or lateral pharyngeal wall or the posterior tonsillar pillars, 403. In yet another example treating PhIX 313, PhX 314, SLN 300, or iSLN 301, comprises treating at least a portion of the oropharyngeal or laryngopharyngeal mucosa 402.

In another embodiment, treating the tissue innervated by LPN 204, PhN 206, comprises treating the parasympathetic and sympathetic pathways to these tissues. In another embodiment, treating at least a portion of tissues innervated by at least one of LPN 204 and PhN 206, comprises treating GPeN 217.

In one embodiment, treating LPN 204, comprises treating the muscles it innervates, including levator veli palatini, palatopharyngeus, and musculus uvulae. In one embodiment treating muscles innervated by LPN 204 comprises treating the muscles with neuromuscular blocking agents such as acetylcholine, suxamethonium, and decamethonium, Aminosteroids, Tetrahydroisoquinoline derivatives, Gallamine and other chemical classes, diester isoquinolinium compounds and bis-benzyltropinium compounds that are bistropinium salts of various diacids, any type of muscle relaxant, Isoquinoline derivatives, Tubocurarine, Atracurium, Cisatracurium, Doxacurium, Metocurine, Mivacurium, Steroid derivatives, Pancuronium, Pipecuronium, Rapacuronium, Rocuronium, Vecuronium, or Botulinum type A toxin.

In one embodiment, the target nerve or tissue is treated using a chemical or pharmaceutical agent. In one embodiment, the chemical or pharmaceutical agent is intended to reduce the hyperreactivity of the target nerves or its associated receptors. In another embodiment, the chemical or pharmaceutical agent is intended to modify the parasympathetic or sympathetic response of the hyperreactive sensory nerve. In yet another embodiment, the chemical or pharmaceutical agent is intended to block the target nerves or its associated receptors.

In one embodiment, the chemical or pharmaceutical agent is injected or otherwise applied to the proximity of the nerve along its course or in the tissue the nerve innervates. In another embodiment, the chemical or pharmaceutical agent is injected or otherwise applied to the proximity of the nerve(s) that carry the parasympathetic or sympathetic fibers to the tissue the sensory nerve innervates. In another embodiment, the chemical or pharmaceutical agent is applied on at least a portion of the tissue the hyperreactive nerve innervates. In one embodiment, the chemical or pharmaceutical agent is formulated with other ingredients to enhance its activity, stability, or deliverability. Formulation ingredients can include, but are not limited to, polymers, viscosity and osmolarity adjustors, stabilizers, humectants, pressurized gas, or mucoadhesive agents.

In one embodiment, the chemical or pharmaceutical agent is in a powder form and is inhaled through the nose or mouth. In yet another example, the chemical or pharmaceutical agent is in a liquid form and is sprayed or otherwise applied through intranasal or intraoral route. In another embodiment, the chemical or pharmaceutical agent is directly applied on the target tissue using an applicator. In one embodiment, the chemical or pharmaceutical agent is in liquid form and is applied by swishing the liquid in the mouth such as oral rinse, mouth rise, or mouth wash. In another embodiment, the chemical or therapeutic agent is embedded within a depot and placed, injected, or implanted in the proximity of the target nerve, or in the tissue the nerve innervates, to achieve sustained release and extend the therapeutic duration. Examples of depots include, but are not limited to, suspensions, liposomes, microparticles, microspheres, polymeric films, in vivo solidifying polymers, in-situ crosslinkable polymers, mucoadhesive polymers, injectable polymers, degradable or nondegradable polymers, pumps such as osmotic pumps. Examples of commercially available long acting corticosteroids include Zilreta® and Kenalog®. Example of commercially available long acting analgesics include Exparel®.

Examples of therapeutics agents include those that act on the sensory nerve or its receptors such as C fibers, cough receptors, mechanoreceptors, and chemoreceptors, to inhibit, reduce, or otherwise modify their function or threshold. Examples of pharmaceutical agents are, but not limited to, TRP channel inhibitors such TRPA1 and TRPV1 antagonist, substance P antagonist, CGRP antagonist, P2×3 antagonist, P2×2 antagonist, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin or other neurotoxin agents, anesthetics, analgesics, corticosteroids, or combination thereof. Other examples of therapeutics agents include those that target the parasympathetic or sympathetic pathways of the hyperreactive nerve. Examples of agents impacting parasympathetic pathway include, but is not limited to, VIP antagonist, muscarinic antagonists, nicotinic antagonists, anticholinergic agents. In another embodiment, the pharmaceutical agents are used to increase the sympathetic activity in the target tissue that is innervated by the hyperreactive sensory neuron. Examples of such pharmaceutical agents include, but is not limited to, beta blockers, beta-2 (β2) agonists and alpha-2 (α2) agonists. Examples of chemicals include, but not limited, to glycerol, phenol, or ethyl alcohol.

In one embodiment, treating LPN 204, PhN 206, PhIX 313, PhX 314, SLN 300, or iSLN 301, comprises surgically sectioning the nerve, its branches, or its ganglia, on at least one point along its course. In another embodiment, treating LPN 204, PhN 206, PhIX 313, PhX 314, SLN 300, or iSLN 301 comprises surgically resecting at least a portion of the tissue these nerves innervate including a portion of nasopharyngeal, oropharyngeal, or laryngopharyngeal mucosa.

In one embodiment to treat LPN 204, chemical or pharmaceutical agent is injected on or in the proximity of at least one of LPN foramina on the palate, as depicted in FIG. 2B. In another embodiment to treat PhN 206, chemical or pharmaceutical agent is injected on or in the proximity of PVF 207 within the nasopharynx. In another embodiment to treat iSLN 301, chemical or pharmaceutical agent is injected on or in the proximity of the area 309, where iSLN 301 pierces through thyroid membrane to reach laryngeal mucosa. In one embodiment pharmaceutical agent include a combination of a corticosteroid and an analgesic. In one example of this embodiment, the corticosteroid is a triamcinolone, and the analgesic is a bupivacaine.

In another embodiment, treating LPN 204, PhN 206, PhIX 313, PhX 314, SLN 300, or iSLN 301, comprises using physical or mechanical methods. In one embodiment, treating LPN 204, PhN 206, PhIX 313, PhX 314, SLN 300, or iSLN 301, comprises applying pressure to the tissues they innervate. In another embodiment, applying pressure to the tissues LPN 204, PhN 206, PhIX 313, PhX 314, or iSLN 301 innervate, comprises using a device.

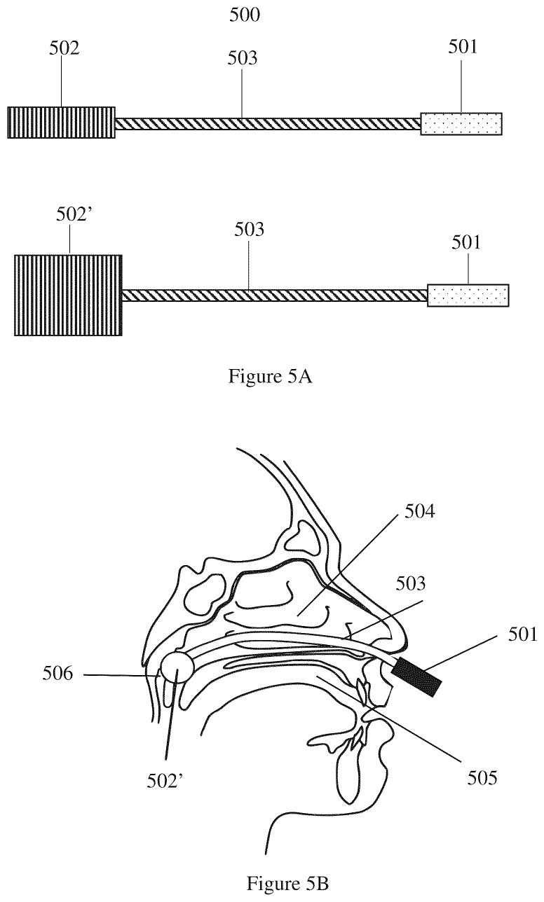

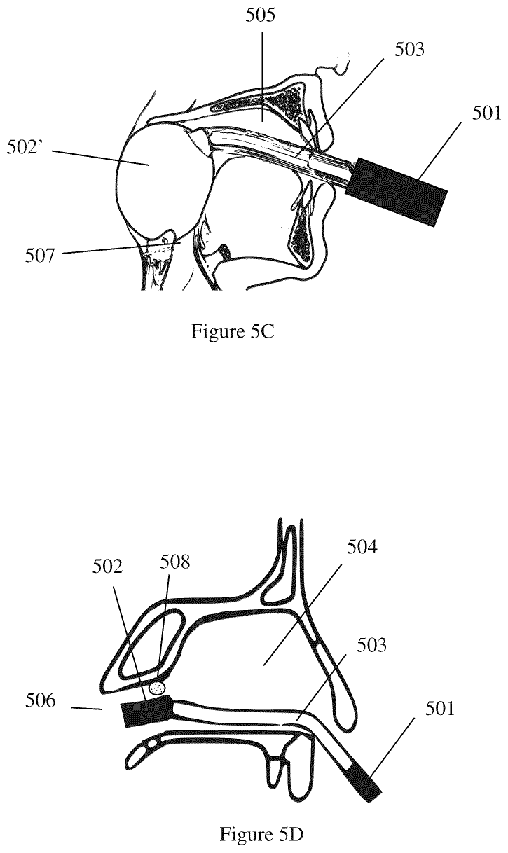

FIG. 5A shows a schematic of a device that can be used to treat a nerve or the tissues the nerve innervates. In one embodiment, the device 500, has a proximal end 501, distal end 502, and elongated shaft 503. The proximal end 501 is used to navigate the distal end 502 to the target location. In one embodiment, the distal end, and elongated shaft are in fluid communication. The proximal end 501 may be configured for coupling to various attachments through cables and/or lumens for delivering and/or removing agents or removing tissue, light sources, optical imaging devices, energy delivery modules, etc. The elongated shaft 503 may also be shaped into an elongate configuration or various curved or arcuate configurations to facilitate delivery and placement of the distal end 502. The shaft 503 may also be configured to be malleable or rigid depending upon the desired treatment conditions.

In one embodiment, distal end can assume a collapsed 502 or an expanded configuration 502′.

In one embodiment, the distal end 502 expansion can produce mechanical force and pressure on surrounding tissue. The force or pressure can impact the function of the cells, neuronal fibers, neuronal receptors, or the blood vessels of the tissue. In one embodiment, device 500 is an expandable balloon catheter.

FIG. 5B depicts the sagittal view of the head including the nasal cavity 504 and oral cavity 505. In one embodiment, the device 500 is used to treat tissues innervated by at least one of LPN 204 and PhN 206. In one embodiment, device 500 is an expandable balloon. In one embodiment, device 500 is navigated through the nasal cavity 504, to reach nasopharynx 506. Once in position, distal end 502 is expanded from collapsed state 502 to expanded state 502′ to apply pressure on the tissues innervated by at least one of LPN 204 and PhN 206. Upon completion of the procedure, distal end 502′ is deflated back to its collapsed state 502 (not shown) before it is navigated out of nasal cavity 504. In another embodiment, device 500 reaches nasopharynx 506, through oral cavity 505.

FIG. 5C depicts the sagittal cross section of the head including oral cavity 505. In one embodiment, the device 500 is used to treat tissues innervated by PhIX 313, PhX 314, SLN 303 or iSLN 301. In one embodiment, device 500 is an expandable balloon. In one embodiment, device 500 is navigated through the oral cavity 505, to reach pharynx 507, which can include at least a portion of oropharynx or laryngopharynx, to treat tissues innervated by PhIX 313, PhX 314, SLN 300 or iSLN. Once in position, distal end 502 is expanded to expanded state 502′ to apply pressure on the surrounding tissues. Upon completion of the procedure, distal end 502′ is deflated back to its collapsed state (not shown), before device 500 is navigated out of oral cavity 505. In another embodiment, device 500 reaches the pharynx 507 through the nasal cavity (not shown).

Examples of commercially available balloon catheter devices that can be used to treat tissues innervated by at least one of LPN 204, PhN 206, PhIX 313, PhX 314, SLN 300 or iSLN 301 include, but not limited to, CRE™ (Boston Scientific), Elation® (Merit Medical, as described in U.S. Pat. Nos. 10,130,797 and 10,086,173), Inspira Air® (Johnson and Johnson, as described in U.S. Pat. No. 9,913,964), Treachealator (DISA Medinotec), Rapid Rhino (Smith and Nephew), EpiStax (Summit Medical), other epistaxis balloons.

In another embodiment, treating at least a LPN 204, PhN 206, PhIX 313, PhX 314, SLN 300 or iSLN 301, or at least a portion of the tissues they innervate, comprises using energy. Examples of energy modalities include but are not limited to radiofrequency energy, pulsed radiofrequency energy, laser, ultrasonic energy, thermal energy (either heat or cryogenic), electrical energy, electromagnetic energy, or light. In one embodiment, the treatment device is a cryogenic device. In one embodiment the energy-based or the cryogenic device comprises an expandable balloon.

In one embodiment, treatment using energy is performed using a device. In one embodiment, the device used for treatment with energy has a similar configuration as device 500 depicted in FIG. 5A, having a proximal end 501, distal end 502, and elongated shaft 503. In one embodiment distal end 502 comprises an energy effector component. Upon reaching the target nerve(s), or the tissue the nerve innervates, the effector component is activated to deliver energy to effect treatment. Upon completion of treatment, the effector component of distal end 502 is deactivated and device 500 is navigated away. In one embodiment, distal end can assume a collapsed 502 and an expanded configuration 502′.

FIG. 5D is schematic depiction of cross section of nasal cavity 504 and nasopharynx 506 and PVF 508. In one embodiment, the device 500 is used to treat a disease. In one embodiment, the distal end 502 of device 500 has an energy effector component.

In one embodiment PhN 206 is treated using an energy-based or cryogenic device. According to this embodiment device 500 is navigated through the nasal cavity to reach nasopharynx 506 and PVF 508. Distal end 502 is then placed on or in proximity of PVF 207 and the effector component is activated to treat all or substantially all nerve fibers passing through PVF. Once treatment is completed, the effector component is deactivated and device 500 is navigated out of the nasal cavity 504. In one embodiment, device 500 is navigated through the nasal cavity 504, to reach nasopharynx 506. In another embodiment, device 500 reaches nasopharynx 506, through oral cavity.

In another embodiment, a portion of nasopharyngeal mucosa is treated using an energy-based or cryogenic device. According to this embodiment device 500 is navigated through the nasal cavity to reach nasopharynx 506 and the distal end 502 is placed on at least a portion of the nasopharyngeal mucosa. Once in position, the effector component of distal end 502 is activated to treat at least a portion of the mucosal lining of nasopharynx 506. Once the desired amount of the nasopharyngeal mucosa is treated, the effector component is deactivated and device 500 is navigated out of the nasal cavity 504. In another embodiment device 500 reaches nasopharynx 506 through the oral cavity (not shown).

In one embodiment LPN 204 is treated using an energy-based or cryogenic device. According to this embodiment, device 500 is navigated through the oral cavity. Distal end 502 is placed on or in proximity of at least one of lesser palatine foramina and the effector component is activated to treat all or substantially all nerve fibers passing through LPF. Once treatment is completed, the effector component is deactivated. Similar steps can be repeated to treat other lesser palatine foramina. Once desired amount of LPN 204 fibers is treated device 500 is navigated out of the oral cavity. In one embodiment, LPF 214 is identified through an imaging mechanism, e.g., a CT scan. In another embodiment, a mucosal flap is raised to obtain closer access to LPF 214.

In another embodiment, a portion of mucosa of the soft palate is treated using an energy-based or cryogenic device. According to this embodiment, device 500 is navigated through the oral cavity, to reach the soft palate. Distal end 502 is then place on at least a portion of the soft palate mucosa and the effector component is activated to treat at least a portion of the mucosa. Once the desired amount of soft palate mucosa is treated, the effector component is deactivated and device 500 is navigated out the oral cavity.

In another embodiment, LPN 204 is treated as it travels through the palatine canal 205 (as depicted in FIG. 2A). In one embodiment, an access to palatine canal 205 is created within the maxillary sinus, distal end 502 is navigated to the proximity of LPN fibers passing through palatine canal 205, the effector component is the activated to treat LPN 204. Once desired amount of LPN 204 fibers are treated device 500 is navigated out of the nasal cavity.

In another embodiment at least a portion of iSLN 301 is treated using energy-based or cryogenic device. In one embodiment, an incision is made within the general vicinity of area 310 as depicted in FIG. 3B. Such incision can be made with a different device, or the treatment device can have a design that allows for creating an incision or puncture in the skin. Treatment device 500 is advanced through the incision to reach the proximity of iSLN 302. Once in position, the effector component of distal end 502 is activated to treat all or substantially all iSLN 301 nerve fibers. Once desired amount of iSLN nerve fibers is treated, the effector component is deactivated and device 500 is navigated out of the body. In some instances, the incision is closed using methods known in the art.

In another embodiment oropharyngeal or laryngopharyngeal tissues innervated by PhIX 313, PhX 314, or iSLN 301 are treated using an energy-based or cryogenic device. FIG. 5E depicts a patient lying in supine position with the mouth open. The tissue over the airway is made transparent to show the trajectory of the device within the airway. Device 500 is advanced through the oral cavity, so the distal end 502 reaches the oropharynx or laryngopharynx. Distal end 502 is placed on the target tissue and the effector component is activated to treat at least a portion of oropharyngeal or laryngopharyngeal mucosa. Once the desired portion of the target mucosa is treated, device 500 is navigated out of the oral cavity. In another embodiment, device 500 is navigated through the nose to reach the pharynx.

The activation mechanism of device 500 may be triggered automatically by a controller. In this configuration the distal end 502 has a sensing or navigation component. Upon sensing and/or confirming the location of the target nerve fibers or tissues, the effector component is activated. Alternatively, the ablation mechanism may be triggered directly by the practitioner.

In one embodiment, treatment with energy comprises ablating the nerve or at least a portion of the tissue the nerve innervates. In another embodiment, treatment with energy comprises stimulating the nerve in a manner that counters its hyperreactivity. In yet another embodiment, treatment with energy comprises reducing the conduction of the nerve without fully ablating it.

In one embodiment, the nerve, or the tissue it innervates is treated by extreme cold. In another embodiment, the nerve, or the tissue it innervates are treated at temperatures in the range of, e.g., −20° C. to −100° C. In one embodiment the device 500 is a cryogenic device.

In one embodiment, device 500 is a cryogenic device and uses the Joules-Thomson (JT) effect to produce the ultra-low temperature. In such a configuration the high-pressure refrigerant gas or liquid is injected through an internal lumen to distal end 502 such that when the gas leaves the internal lumen, it expands into an outer lumen causing a drop in temperature. The exhaust gas is then released back to the environment. In another embodiment, the refrigerant gas used to produce the JT effect can also be used to expand the distal end of the device when the distal end is designed to be expandable.

In another embodiment, device 500 sprays the refrigerant gas or liquid directly on the target tissue. In this embodiment proximal end of the device 501, shaft 503, and distal end 502 are in fluid communication (e.g., liquid and/or gas). The proximal end 502 is connected to a source of pressurized refrigerant gas or liquid. Once at the target location, the refrigerant gas or liquid is sprayed directly on the tissue through distal end 502. In one embodiment, the distal end 502 is designed to optimize spray pattern and size.

In another embodiment a principle other than the JT system is used to cool distal end 502 of device 500.

In one embodiment, the cryogenic device 500 uses nitrous oxide as the refrigerant gas. In another embodiment the cryogenic device 500 uses carbon dioxide as the refrigerant gas. In another embodiment the cryogenic device 500 uses any chlorofluorocarbon, hydrochlorofluorocarbon, hydrofluorocarbon or any mixtures thereof as refrigerant. In yet another embodiment the cryogenic device 500 uses liquid nitrogen as refrigerant.

In one embodiment the treatment is done under direct visualization. In other embodiments the treatment is performed under other forms of visualization including, but not limited, to endoscopic, bronchoscopic, fluoroscopic, or ultrasound visualization. In other embodiments, other methods of visualization known in the art is used to assist in performing the treatment. In one embodiment a navigation tool is used to identify and facilitate access to the target tissue.

Examples of energy-based or cryogenic devices that can be used to treat a disease according to this disclosure include Accurian (Medtronic), G4 RF (Boston Scientific), Coolief RF (Avanos medical), Owl® (Diros Tech), Rhinaer® (Aerin medical), Visual Ice™ (Boston Scientific), cryoICE and Cryophere (AtriCure), Iovera (Myoscience), PainBlocker® (Epimed), AveCure (Medwave), Barrx® (Medtronic), Coblator (Arthrocare), Neuromark® (Neurent), Clarifix (Arrinex), Erbecryo® (Erbe), C2 Cryoballoon™ (Pentax Medical), Trufreeze® (Steris), RejuvenAir (CSA medical), Alair™ (Boston Scientific). Prior to treatment, the physician might conduct at least one diagnostic procedure to ensure of the suitability of the treatment target. In one embodiment, diagnostic step includes injecting or applying an anesthetic or another blocking agent, to the target location. In another embodiment, the diagnostic step might include examining the function of a sensory nerve using nerve stimulators, nerve visualizers or other methods known in the art to examine the function of a nerve or its receptors. In yet another embodiment, diagnosis is made by comparing the field of innervation of a given nerve is against a patient's symptoms and other comorbid diseases present in the patient. As an example, cooccurrence of sleep apnea and at least one symptom of otologic dysfunction, bruxism, or nasal symptoms, can point to PhN or LPN as one culprit. As another example, cooccurrence of sleep apnea, swallowing dysfunction, GI symptoms or lower respiratory symptoms can point to iSLN as the main culprit. In yet another example, cooccurrence of heart failure and sleep apnea can point to iSLN as the cause. In one embodiment, a diagnostic tool can be developed in form of a software, for example using artificial intelligence, to match symptoms to innervation patterns.

During the procedure, methods know in the art can be used to visualize the target nerve. As nerves or their branches generally follow blood vessels in the airway mucosa, in one embodiment, methods known in the art to visualize arteries and blood vessels can be used to identify the approximate location of a nerve. Examples of methods used to visualize arteries include, but are not limited to, light including visible light, Magnetic resonance angiography, ultrasonography, sonography, dual red imaging, narrow band imaging, or computerized tomography.

### Jaw Movement Disorders, Bruxism

In one embodiment, at least one of LPN 204, GPN 212, and NPN 210 is treated to treat or reduce symptoms of disorders such as, but not limited to, bruxism, orofacial muscle pain, myalgia (Muscle Pain), temporomandibular disorders, endinosis or enthesopathy, myositis, spasm, jaw movement disorders such as orofacial/oromandibular dyskinesia and dystonia, oral mucosal inflammatory disorders, palatal inflammatory disorders such as palatal torus, abscess or cyst, gingival or periodontal inflammatory diseases such as periodontitis and gingivitis.

In one embodiment, treating LPN 204 comprises treating it anywhere along its course, ganglia or branches. In one embodiment LPN 204 is treated at its foramina, LPF 214, as it enters the oral cavity. In another embodiment LPN is treated on its course through the bony canal 205 from SPG 203 to LPF 214. In another embodiment LPN 104 is treated along its course on or in the soft palate 201. In one embodiment, LPN 204 is treated intranasally. In another embodiment, LPN 204 is treated intraorally.

In one embodiment, treating GPN 212 comprises treating it anywhere along its course, its ganglia, or its branches. In one embodiment GPN 212 is treated at its foramina, GPF 215, as it enters the oral cavity. In another embodiment GPN is treated on its course through the bony canal 205 from SPG 203 to GPF 215. In another embodiment GPN 212 is treated along its course on or in the hard palate 213. In one embodiment, GPN 212 is treated intranasally through the maxillary sinus. In another embodiment, GPN 212 is treated intraorally.

In one embodiment, treating NPN 210 comprises treating it anywhere along its course, its ganglia, or its branches. In one embodiment NPN 210 is treated at its foramina, IF 216, as it enters the oral cavity. In another embodiment NPN 210 is treated on its course in the nasal cavity 200 to IF 216. In another embodiment NPN 210 is treated along its course on or in the hard palate 213. In one embodiment, NPN 210 is treated intranasally. In another embodiment, NPN 210 is treated intraorally.

In one embodiment, treating at least one of LPN 204, NPN 210, or GPN 212, comprises treating at least a portion of mucosa of nasal septum or the mucosa of the soft or hard palate.

In another embodiment treating the tissue innervated by LPN 204, NPN 210, or GPN 212 comprises treating the parasympathetic and sympathetic fibers that are present in the tissues they innervate. In another embodiment, treating LPN 204, NPN 210, or GPN 212 comprises treating GPeN 217.

In one embodiment, the target nerve is treated with a chemical or pharmaceutical agent. In one embodiment, the chemical or pharmaceutical agent is intended to reduce or hyperreactivity of the target nerves or its associated receptors. In another embodiment, the chemical or pharmaceutical agent is intended to modify the parasympathetic or sympathetic response of the hyperreactive sensory nerve.

In one embodiment, the chemical or pharmaceutical agent is injected or otherwise applied to the proximity of the nerve along its course or in the tissue the nerve innervates. In another example, the chemical or pharmaceutical agent is injected or otherwise applied to the proximity of the nerve(s) that carry the parasympathetic or sympathetic fibers to the tissue the sensory nerve innervates. In another embodiment, the chemical or pharmaceutical agent is applied on the tissue the hyperreactive nerve innervates. In one embodiment the chemical or pharmaceutical agent is formulated with other ingredients to enhance its activity, stability, or deliverability. Formulation ingredients can include but are not limited to polymers, viscosity and osmolarity adjustors, stabilizers, humectants, pressurized gas, or mucoadhesive agents.

In one embodiment, the chemical or pharmaceutical agent is in a powder form and is inhaled through the nose or mouth. In yet another example, the chemical or pharmaceutical agent is in a liquid form and is sprayed or otherwise applied through intranasal or intraoral route. In another embodiment, the chemical or pharmaceutical agent is directly applied on the target tissue. In one embodiment, the chemical or pharmaceutical agent is in liquid form and is applied by swishing the liquid in the mouth such as oral rinse, mouth rise, or mouth wash. In another embodiment, the chemical or therapeutic agent is embedded within a depot and placed, injected, or implanted in the proximity of the target nerve, or in the tissue it innervates, to achieve sustained release and extend the therapeutic duration. Examples of depots includes but is not limited to liposomes, microparticles, microspheres, polymeric films, in vivo solidifying polymers, in-situ crosslinkable polymers, mucoadhesive polymers, injectable polymers, degradable or nondegradable polymers, pumps such as osmotic pumps. Examples of commercially available long corticosteroids include Zilreta® and Kenalog®. Example of commercialy available long acting analgesics include Exparel®.

Examples of therapeutics agents include those that act on the sensory nerve receptors such as C fibers, cough receptors, mechanoreceptors, and chemoreceptors to inhibit, reduce, or otherwise modify their function. Examples of pharmaceutical agents are but not limited to, TRP channel inhibitors such TRPA1 and TRPV1 antagonist, substance P antagonist, CGRP antagonist, P2×3 antagonist, P2×2 antagonist, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin or other neurotoxin agents, anesthetics, analgesics, corticosteroids, or combination thereof. Other examples of therapeutics agents include those that target the parasympathetic or sympathetic pathways of the hyperreactive nerve. Examples of agents impacting parasympathetic pathway include, but is not limited to, VIP antagonist, muscarinic antagonists, nicotinic antagonists, anticholinergic agents. In another embodiment, the pharmaceutical agents are used to increase the sympathetic activity in the target tissue that is innervated by the hyperreactive sensory neuron. Examples of such pharmaceutical agents include, but is not limited to, beta blockers, beta-2 (β2) agonists and alpha-2 (α2) agonists. Examples of chemicals include, but not limited, to glycerol, phenol, or ethyl alcohol.

In one embodiment to treat LPN 204, chemical or pharmaceutical agent is injected on or in the proximity of at least one of LPN foramina on the palate, as depicted in FIG. 2B. In another embodiment to treat GPN 212, chemical or pharmaceutical agent is injected on or in the proximity of at least one of greater palatine foramina on the palate, as depicted in FIG. 2B. In another embodiment to treat NPN 210, chemical or pharmaceutical agent is injected on or in the proximity of at least incisor foramen on the palate, as depicted in FIG. 2B. In yet another embodiment to treat NPN 210, chemical or pharmaceutical agent is injected on or in the proximity of NPN course on the nasal septum. In one embodiment the pharmaceutical agent is a corticosteroid. In another embodiment the pharmaceutical agent is an analgesic. In another embodiment the pharmaceutical agent is a mixture of a corticosteroid and an analgesic.

In another embodiment, treating at least one of LPN 204, NPN 210, or GPN 212, comprises using physical or mechanical methods. In one embodiment, treating LPN 204, NPN 210, or GPN 212, comprises applying pressure to the tissues they innervate. In another embodiment, treating LPN 204, NPN 210, or GPN 212 comprises applying a physical barrier on the tissue they innervate to prevent the receptors from contact with chemical or physical stimuli.

In one embodiment at least a portion of the tissue innervated by NPN 210 or GPN 212, is treated using a physical barrier. In one embodiment a physical barrier is applied over at least a portion of hard palate to prevent the mechanoreceptors innervating the hard palate to be stimulated.

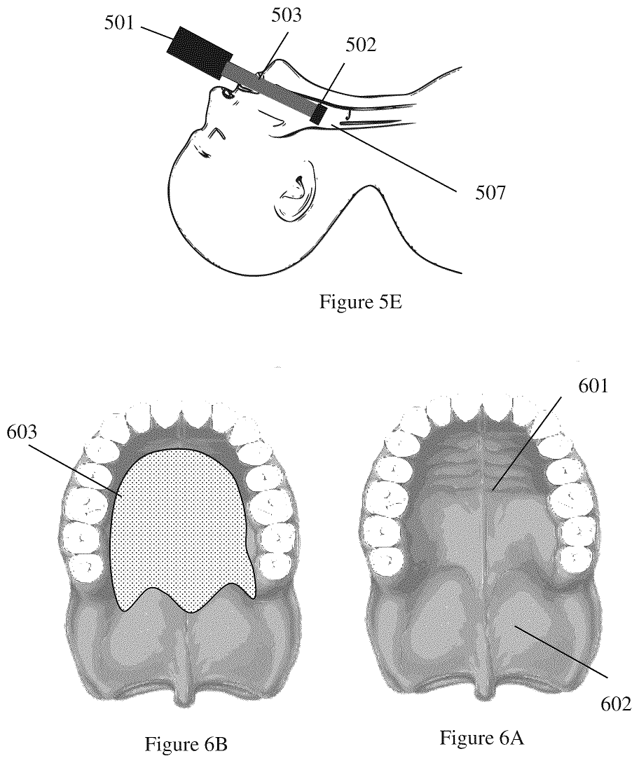

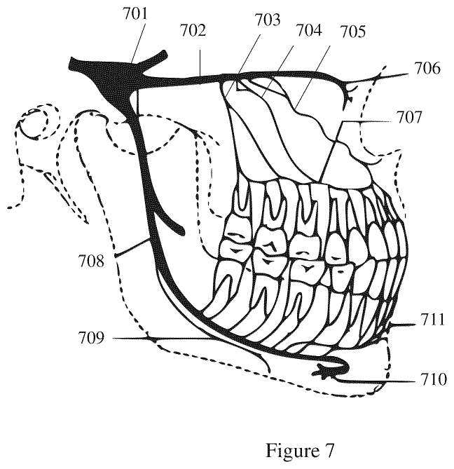

FIG. 6A shows the traverse cross section of the roof of the oral cavity comprising hard palate 601 and soft palate 602. FIG. 6B shows the hard palate 601 is covered by physical barrier 603. In one embodiment, physical barrier 603 can be made to cover at least a portion of hard palate 601. In another embodiment, physical barrier 603 is made of, but not limited to, polymers, plastics, elastics, hydrogels, ceramics or metals. In one embodiment, physical barrier 603 is made of at least one metal wire. In another embodiment, physical barrier 603 stays in place by an anchoring mechanism. In another embodiment, the anchoring mechanism uses at least one tooth to anchor physical barrier 603 in place. In yet another embodiment, physical barrier 603 also covers at least one tooth. In another embodiment, physical barrier 603 is made to anatomically match the patient.

In another embodiment, treating LPN 204, NPN 210, or GPN 212, or at least a portion of the tissue they innervate, comprises using energy. Examples of energy modalities include but are not limited to radiofrequency energy, pulsed radiofrequency energy, laser, ultrasonic energy, thermal energy (either heat or cryogenic), electrical energy, electromagnetic energy, or light.

In one embodiment, treatment using energy is performed using an effector device. In one embodiment, the device used for treatment has a similar configuration as device 500 depicted in FIG. 5A, having a proximal end 501, distal end 502, and elongated shaft 503. In one embodiment distal end 502 comprises an energy effector component. Upon reaching the target nerve(s), or the tissue the nerve innervates, the effector component is activated to deliver energy to effect treatment. Upon completion of treatment, the effector component of distal end 502 is deactivated and device 500 is navigated away from the target.

In one embodiment, treatment with energy comprises ablating at least one of LPN 204, NPN 210, or GPN 212 or at least a portion of the tissue they nerve innervate. In another embodiment, treatment with energy comprises stimulating the nerve in a manner that counters its hyperreactivity. In yet another embodiment, treatment with energy comprises reducing the conduction of the nerve.

In one embodiment, at least one of LPN 204, NPN 210, or GPN 212, or the tissue it innervates is treated by device 500. In one embodiment, device 500 is a cryogenic device that uses temperatures within the range of, e.g., −20° C. to −100° C. to ablate at least one of LPN 204, NPN 210, or GPN 212, or at least a portion of the tissue they innervate.

In one embodiment, the cryogenic device 500 uses the Joules-Thomson (JT) effect to produce the ultra-low temperature. In another embodiment, cryogenic device 500 sprays the refrigerant gas or liquid directly on the target tissue. In another embodiment a principle other than the JT system is used to cool the distal end of the device to treat tissue.

In one embodiment the cryogenic device 500 uses nitrous oxide as the refrigerant gas. In another embodiment the cryogenic device 500 uses carbon dioxide as the refrigerant gas. In another embodiment the cryogenic device 500 uses any chlorofluorocarbon, hydrochlorofluorocarbon, hydrofluorocarbon or any mixtures thereof as refrigerant. In yet another embodiment the cryogenic device 500 uses liquid nitrogen as refrigerant.

In one embodiment LPN 204 is treated using an energy-based or cryogenic device. According to this embodiment, device 500 is navigated through the oral cavity. Distal end 502 is placed on or in proximity of at least one of lesser palatine foramina and the effector component is activated to treat all or substantially all nerve fibers passing through LPF. Once treatment is completed, the effector component is deactivated. Similar steps can be repeated to treat other lesser palatine foramina. Once desired amount of LPN 204 fibers is treated device 500 is navigated out of the oral cavity. In one embodiment, LPF 214 is identified through an imaging mechanism, e.g., a CT scan. In another embodiment, a mucosal flap is raised to obtain closer access to LPF 214.

In one embodiment GPN 212 is treated using an energy-based or cryogenic device. According to this embodiment, device 500 is navigated through the oral cavity. Distal end 502 is placed on or in proximity of at least one of greater palatine foramina and the effector component is activated to treat all or substantially all nerve fibers passing through GPF. Once treatment is completed, the effector component is deactivated. Similar steps can be repeated to treat other greater palatine foramina. Once desired amount of GPN 212 fibers is treated device 500 is navigated out of the oral cavity. In one embodiment, GPF is identified through an imaging mechanism, e.g., a CT scan. In another embodiment, a mucosal flap is raised to obtain closer access to GPF.

In one embodiment NPN 210 is treated using an energy-based or cryogenic device. According to this embodiment, device 500 is navigated through the oral cavity. Distal end 502 is placed on or in proximity of incisor foramen and the effector component is activated to treat all or substantially all nerve fibers passing through IF. Once treatment is completed, the effector component is deactivated. Once desired amount of NPN 210 fibers is treated device 500 is navigated out of the oral cavity.

In another embodiment a portion of the mucosa of hard or soft palate is treated using an energy-based or cryogenic device. In this embodiment, device 500 is navigated through the oral cavity, distal end 502 is placed on the target tissue and the effector component is activated. Once desired portion of the mucosa is treated the effector component is deactivated and device 500 is navigated out of the oral cavity.

In another embodiment to treat NPN 210 a portion of the mucosa of the nasal septum is treated using an energy-based or cryogenic device. In this embodiment, device 500 is navigated through the nasal cavity, distal end 502 is placed on the target tissue and the effector component is activated. Once desired portion of the mucosa is treated the effector component is deactivated and device 500 is navigated out of the nasal cavity.

In one embodiment the treatment is done under direct visualization. In other embodiments the treatment is performed under other forms of visualization including, but not limited, to endoscopic, bronchoscopic, fluoroscopic, or ultrasound visualization. In other embodiments, other methods of visualization known in the art is used to assist in performing the treatment. In one embodiment a navigation tool is used to identify and facilitate access to the target tissue.

Prior to treatment, the physician might conduct at least one diagnostic procedure to ensure of the suitability of the treatment target. In one embodiment, diagnostic step includes injecting or applying an anesthetic or another blocking agent, to the target location. In another embodiment, the diagnostic step might include examining the function of a sensory nerve using nerve stimulators, nerve visualizers or other methods known in the art to examine the function of a nerve or its receptors. In yet another embodiment, diagnosis is made by comparing the field of innervation of a given nerve is against a patient's symptoms and other comorbid diseases present in the patient. In one embodiment, a diagnostic tool can be developed in form of a software, for example using artificial intelligence, to match symptoms to innervation patterns.

During the procedure, methods know in the art can be used to visualize the target nerve. As nerves or their branches generally follow blood vessels in the airway mucosa, in one embodiment, methods known in the art to visualize arteries and blood vessels can be used to identify the approximate location of a nerve. Examples of methods used to visualize arteries include, but are not limited to, light including visible light, Magnetic resonance angiography, ultrasonography, sonography, dual red imaging, narrow band imaging, or computerized tomography.

### Periodontitis and Gingivitis

FIG. 7 shows the schematic representation of the upper and lower jaws their sensory innervation. Maxillary nerve 702 leaves trigeminal ganglion 701 before branching into posterior superior alveolar nerve (PSAN) 703, middle superior alveolar nerve (MSAN) 704, anterior superior alveolar nerve (ASAN) 704 before exiting the infraorbital foramen 706. Branches of superior alveolar nerve descend through the maxillary sinus (not specified in the figure) to form superior dental plexus 707 that supplies the innervation the roots of the upper teeth, maxillary gums and buccal mucosa. Inferior alveolar nerve (IAN) 708, is a branch of mandibular nerve (not shown). It issues the mylohyoid nerve 709 before entering the mandibular canal (not shown). Passing through the canal, it issues sensory branches for the molar and second premolar teeth. It terminates by splitting into its two terminal branches: the mental nerve 710, and the incisive branch 711.

In one embodiment, at least one of PSAN 703, MSAN 704, ASAN 705, IAN 708 is treated to treat or reduce symptoms of disorders such as, but not limited to, bruxism, orofacial muscle pain, myalgia (Muscle Pain), temporomandibular disorders, endinosis or enthesopathy, myositis, spasm, jaw movement disorders such as orofacial/oromandibular dyskinesia and dystonia, oral mucosal inflammatory disorders, palatal inflammatory disorders such as palatal torus, abscess or cyst, gingival or periodontal inflammatory diseases such as periodontitis and gingivitis.

In one embodiment treating PSAN 703, MSAN 704, or ASAN 705 comprises treating the nerves as they course through the maxillary sinus. In one embodiment treating at least one of PSAN 703, MSAN 704, or ASAN 705 includes treating at least a portion of maxillary sinus mucosa. In one embodiment treating PSAN 703, MSAN 704, or ASAN 705 comprises treating the superior dental plexus. In another embodiment treating PSAN 703, MSAN 704, or ASAN 705 comprises treating the sphenopalatine ganglion.

In one embodiment treating IAN 708 comprises treating it anywhere along its course. In one embodiment treating IAN 708 comprises treating incisor branch 711. In another embodiment treating IAN 708 comprising treating at least a portion of mandibular mucosa. In another embodiment treating IAN 708 comprises treating tympanic branch of the glossopharyngeal nerve.

In one embodiment, the target nerve is treated with a chemical or pharmaceutical agent. In one embodiment, the chemical or pharmaceutical agent is intended to reduce or hyperreactivity of the target nerves or its associated receptors. In another embodiment, the chemical or pharmaceutical agent is intended to modify the parasympathetic or sympathetic response of the hyperreactive sensory nerve.

In one embodiment, the chemical or pharmaceutical agent is injected or otherwise applied to the proximity of the nerve along its course or in the tissue the nerve innervates. In another example, the chemical or pharmaceutical agent is injected or otherwise applied to the proximity of the nerve(s) that carry the parasympathetic or sympathetic fibers to the tissue the sensory nerve innervates. In another embodiment, the chemical or pharmaceutical agent is applied on the tissue the hyperreactive nerve innervates. In one embodiment the chemical or pharmaceutical agent is formulated with other ingredients to enhance its activity, stability, or deliverability. Formulation ingredients can include but are not limited to polymers, viscosity and osmolarity adjustors, stabilizers, humectants, pressurized gas, or mucoadhesive agents.

In one embodiment, the chemical or pharmaceutical agent is in a powder form and is inhaled through the nose or mouth. In yet another example, the chemical or pharmaceutical agent is in a liquid form and is sprayed or otherwise applied through intranasal or intraoral route. In another embodiment, the chemical or pharmaceutical agent is directly applied on the target tissue. In one embodiment, the chemical or pharmaceutical agent is in liquid form and is applied by swishing the liquid in the mouth such as oral rinse, mouth rise, or mouth wash. In another embodiment, the chemical or therapeutic agent is embedded within a depot and placed, injected, or implanted in the proximity of the target nerve, or in the tissue it innervates, to achieve sustained release and extend the therapeutic duration. Examples of depots includes but is not limited to liposomes, microparticles, microspheres, polymeric films, in vivo solidifying polymers, in-situ crosslinkable polymers, mucoadhesive polymers, injectable polymers, degradable or nondegradable polymers, pumps such as osmotic pumps. Examples of commercially available long corticosteroids include Zilreta® and Kenalog®. Example of commercialy available long acting analgesics include Exparel®.

Examples of therapeutics agents include those that act on the sensory nerve receptors such as C fibers, cough receptors, mechanoreceptors, and chemoreceptors to inhibit, reduce, or otherwise modify their function. Examples of pharmaceutical agents are but not limited to, TRP channel inhibitors such TRPA1 and TRPV1 antagonist, substance P antagonist, CGRP antagonist, P2×3 antagonist, P2×2 antagonist, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin or other neurotoxin agents, anesthetics, analgesics, corticosteroids, or combination thereof. Other examples of therapeutics agents include those that target the parasympathetic or sympathetic pathways of the hyperreactive nerve. Examples of agents impacting parasympathetic pathway include, but is not limited to, VIP antagonist, muscarinic antagonists, nicotinic antagonists, anticholinergic agents. In another embodiment, the pharmaceutical agents are used to increase the sympathetic activity in the target tissue that is innervated by the hyperreactive sensory neuron. Examples of such pharmaceutical agents include, but is not limited to, beta blockers, beta-2 (β2) agonists and alpha-2 (α2) agonists. Examples of chemicals include, but not limited, to glycerol, phenol, or ethyl alcohol.

In another embodiment, treating PSAN 703, MSAN 704, ASAN 705, IAN 708, or at least a portion of the tissue they innervate, comprises using energy. Examples of energy modalities include but are not limited to radiofrequency energy, pulsed radiofrequency energy, laser, ultrasonic energy, thermal energy (either heat or cryogenic), electrical energy, electromagnetic energy, or light.

In one embodiment, treatment using energy is performed using an effector device. In one embodiment, the device used for treatment has a similar configuration as device 500 depicted in FIG. 5A, having a proximal end 501, distal end 502, and elongated shaft 503. In one embodiment distal end 502 comprises an energy effector component. Upon reaching the target nerve(s), or the tissue the nerve innervates, the effector component is activated to deliver energy to effect treatment. Upon completion of treatment, the effector component of distal end 502 is deactivated and device 500 is navigated away from the target.

In one embodiment, treatment with energy comprises ablating at least one of PSAN 703, MSAN 704, ASAN 705, IAN 708 or at least a portion of the tissue they nerve innervate. In another embodiment, treatment with energy comprises stimulating the nerve in a manner that counters its hyperreactivity. In yet another embodiment, treatment with energy comprises reducing the conduction of the nerve.

In one embodiment, at least one of PSAN 703, MSAN 704, ASAN 705, IAN 708, or the tissue it innervates is treated by device 500. In one embodiment, device 500 is a cryogenic device that uses temperatures within the range of, e.g., −20° C. to −100° C. to ablate at least one of LPN 204, NPN 210, or GPN 212, or at least a portion of the tissue they innervate.

In one embodiment, the cryogenic device 500 uses the Joules-Thomson (JT) effect to produce the ultra-low temperature. In another embodiment, cryogenic device 500 sprays the refrigerant gas or liquid directly on the target tissue. In another embodiment a principle other than the JT system is used to cool the distal end of the device to treat tissue.

In one embodiment the cryogenic device 500 uses nitrous oxide as the refrigerant gas. In another embodiment the cryogenic device 500 uses carbon dioxide as the refrigerant gas. In another embodiment the cryogenic device 500 uses any chlorofluorocarbon, hydrochlorofluorocarbon, hydrofluorocarbon or any mixtures thereof as refrigerant. In yet another embodiment the cryogenic device 500 uses liquid nitrogen as refrigerant.

In one embodiment PSAN 703, MSAN 704, ASAN 705 are treated using an energy-based or cryogenic device. According to this embodiment, device 500 is navigated through the nasal cavity. Distal end 502 is navigated to the maxillary sinus. The effector component is activated to treat all or substantially all nerve embedded within maxillary sinus mucosa. Once treatment is completed, the effector component is deactivated.

In one embodiment the treatment is done under direct visualization. In other embodiments the treatment is performed under other forms of visualization including, but not limited, to endoscopic, bronchoscopic, fluoroscopic, or ultrasound visualization. In other embodiments, other methods of visualization known in the art is used to assist in performing the treatment. In one embodiment a navigation tool is used to identify and facilitate access to the target tissue.

Prior to treatment, the physician might conduct at least one diagnostic procedure to ensure of the suitability of the treatment target. In one embodiment, diagnostic step includes injecting or applying an anesthetic or another blocking agent, to the target location. In another embodiment, the diagnostic step might include examining the function of a sensory nerve using nerve stimulators, nerve visualizers or other methods known in the art to examine the function of a nerve or its receptors. In yet another embodiment, diagnosis is made by comparing the field of innervation of a given nerve is against a patient's symptoms and other comorbid diseases present in the patient. In one embodiment, a diagnostic tool can be developed in form of a software, for example using artificial intelligence, to match symptoms to innervation patterns.

During the procedure, methods know in the art can be used to visualize the target nerve. As nerves or their branches generally follow blood vessels in the airway mucosa, in one embodiment, methods known in the art to visualize arteries and blood vessels can be used to identify the approximate location of a nerve. Examples of methods used to visualize arteries include, but are not limited to, light including visible light, Magnetic resonance angiography, ultrasonography, sonography, dual red imaging, narrow band imaging, or computerized tomography.

### Methods

### Treating the Nerve

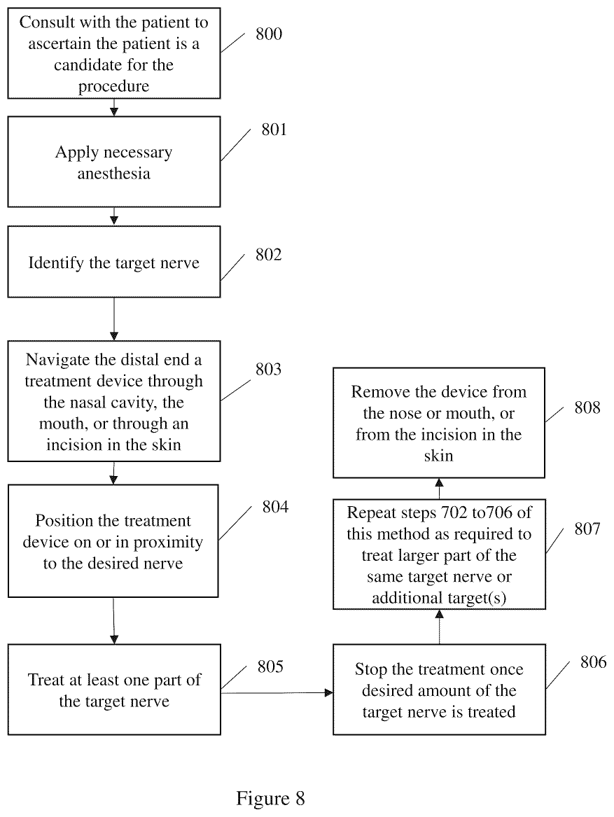

Step by step of one method prescribed for treating diseases is depicted in the flow diagram of FIG. 8:
a. Consult with the patient and ascertain that the patient is a candidate for the current procedure 800,
b. Apply necessary anesthesia to the region of tissue to be treated or put the patient under general anesthesia 801,
c. Identify the target nerve 802,
d. Navigate the distal end of a treatment device through the nasal cavity, the mouth, or through an incision in the skin 803,
e. Position the treatment device on or in proximity to the desired nerve 804,
f. Treat at least one part of the target nerve 805,
g. Stop the treatment once desired amount of the target nerve is treated 806,
h. Repeat steps 802 to 806 of this method as required to treat larger part of the same target nerve or additional target(s) 807,
i. Remove the device from the nose or mouth, or from the incision in the skin 808.

In the method described above, some of the individual steps may be omitted entirely or applied in an alternative order.

In one embodiment of the method above, more than one nerve is treated. In another embodiment and with respect to the identification of the appropriate target tissue, temporary anesthetics can be applied to the target to observe the impact of the nerve block.

In one embodiment of the method, diseases to be treated include symptoms associated sleep disordered breathing, snoring, obstructive sleep apnea (OSA), heart failure and its associated symptoms, hypertension, metabolic diseases such as diabetes, insulin resistance, arrythmia, obesity, nasal and nasopharyngeal symptoms, chronic cough, otologic conditions such as tinnitus, eustachian tube dysfunction, vertigo, Meniere's disease, hearing loss, imbalance, lymphoid tissue hypertrophy, jaw movement disorders such as bruxism, temporomandibular diseases, oral tissue inflammatory diseases such as gingivitis and periodontitis.

In another embodiment target nerve include at least a portion of NPN, LPN, PhN, GPN, PhIX, PhX, SLN, or iSLN.

In one embodiment the treatment device is a drug delivery device, to deliver pharmaceutical or chemical agents to the target nerve.

In another embodiment the treatment device uses energy such as radiofrequency, microwave, heat, or pulsed radiofrequency to treat the nerve. In another embodiment the treatment device is a cryogenic device.

In one embodiment, treatment device is used to ablate the target nerve. In another embodiment, the treatment device uses energy to reduce or otherwise modify the function of the target nerve.

In one embodiment of the method described herein, the step to identify the target nerve or nerves can include using a nerve tissue visualization aid (e.g., dyes) or other techniques to distinguish nerve tissue from surrounding tissues. In another embodiment, the step to identify target nerve includes methods of nerve stimulation and/or nerve monitoring techniques commonly known in the art. In another method techniques to access the function of a sensory nerve is used to identify the target tissue. In yet another embodiment, methods known in the art is used to visualize the blood vessels and arteries and by association the target nerve.

In one embodiment of the method of the current application, the patient is diagnosed with sleep apnea by the physician and a candidate for current procedure. A device used in the procedure is a cryoprobe, capable to reach temperatures between −20° C. to −100° C., using nitrous oxide as cryogen and by employing the Joules Thompson principal. The surgeon uses visualization with or without the help of a navigation instrument to identify at least a portion of at least a LPN, a PhN, or an iSLN. The cryoprobe is then navigated through the nasal cavity, through the mouth, or through an incision on the skin to reach the desired part of the nerve to be ablated. The distal end of the cryoprobe is placed on the nerve and the ablation mechanism is triggered by allowing the cryogen to flow though the cryoprobe. The nerve is then frozen for a period of about 10 seconds to 60 seconds. The flow of cryogen is stopped, and the distal end is allowed to thaw. Once the distal end is released from tissue, the ablation procedure might be repeated on additional locations until desired length of nerve(s) is ablated. Once the ablation is complete, the cryoprobe is navigated out of the nose or the mouth.

In another embodiment of the method above, the patient is diagnosed hypertension by the physician and a candidate for current procedure. A device used in the procedure is a cryoprobe, capable to reach temperatures between −20° C. to −100° C., using nitrous oxide as cryogen and by employing the Joules Thompson effect. The surgeon uses visualization with or without the help of a navigation instrument to identify SLN or iSLN. To access the nerve, an incision is made on the skin approximately between the greater horn of the hyoid bone and the thyroid cartilage. The distal end of the cryoprobe is navigated through the incision and is placed in the proximity of iSLN and the ablation mechanism is triggered by allowing the cryogen to flow though the cryoprobe. The nerve is then frozen for a period of about 10 seconds to 60 seconds. The flow of cryogen is stopped, and the distal end is allowed to thaw. Once the distal end is released from tissue, the ablation procedure might be repeated on additional locations until desired length of nerve(s) is ablated. Once the ablation is complete, the cryoprobe is navigated out of the incision and the incision is closed.

In another embodiment or the method above, the patient is diagnosed with heart failure. In another embodiment, the patient is diagnosed with a metabolic disease such as diabetes or insulin resistance. In yet another embodiment, the patient is diagnosed with SBD. In one embodiment, the patient is diagnosed with chronic cough. In one embodiment, in addition to iSLN, at least a portion of PhN is also ablated. In another embodiment, the target nerve to be ablated is at least a portion of a PhIX, or a PhX. In another embodiment, the treatment device uses radiofrequency energy to ablate at least a portion of iSLN. In other embodiments, the treatment device uses other sources of energy to ablate at least a portion of iSLN.

In one method of the current application, the patient is diagnosed with eustachian tube dysfunction by the physician and a candidate for current procedure. The device used in the procedure is a cryoprobe, capable to reach temperatures between −20° C. to −100° C., using nitrous oxide as cryogen and by employing the Joules Thompson effect. The surgeon uses visualization with or without the help of a navigation instrument to identify at least a portion of PhN or its foramina. The cryoprobe is then navigated through the nasal cavity or through the mouth to reach the desired part of the nerve to be ablated. The distal end of the cryoprobe is placed on or in the proximity of PVF and the ablation mechanism is triggered by allowing the cryogen to flow though the cryoprobe. The PhN nerve fibers going through PVF are frozen for a period of about 10 seconds to 60 seconds. The flow of cryogen is stopped, and the distal end is allowed to thaw. Once the distal end is released from tissue, the ablation procedure might be repeated. Once the ablation is complete, the cryoprobe is navigated out of the nose or the mouth. In another embodiment the patient is diagnosed with hypertension. In yet another embodiment, the patient is diagnosed with SBD. In another embodiment, the treatment device uses radiofrequency energy to ablate at least a portion of PhN. In other embodiments, the treatment device uses other sources of energy to ablate at least a portion of PhN.

In one method of the current application, the patient is diagnosed with a jaw movement disorder by the physician and a candidate for current procedure. The device used in the procedure is a cryoprobe, capable to reach temperatures between −20° C. to −100° C., using nitrous oxide as cryogen and by employing the Joules Thompson effect. The surgeon uses visualization method with or without the help of a navigation instrument to identify at least a portion of at least a GPN, a NPN, or a LPN or their foramina. The cryoprobe is then navigated through the mouth to reach the desired location. If the nerve is too deep within the tissue, an incision might be made to allow for the closer contact between the nerve and the treatment device. The distal end of the cryoprobe is placed on the tissue and the ablation mechanism is triggered by allowing the cryogen to flow though the cryoprobe. The tissue is then frozen for a period of about 10 seconds to 60 seconds. The flow of cryogen is stopped, and the distal end is allowed to thaw. Once the distal end is released from tissue, the ablation procedure might be repeated on additional locations until desired surface area is ablated. Once the ablation is complete, the cryoprobe is navigated out of the mouth. In another embodiment the patient is diagnosed with temporomandibular joint disorder. In yet another embodiment, the patient is diagnosed with oral mucosal inflammatory disease such as gingivitis or periodontitis. In another embodiment, the treatment device uses radiofrequency energy to ablate at least a portion of a GPN, a NPN, or a LPN. In other embodiments, the treatment device uses other sources of energy to ablate at least a portion of at least a portion of a GPN, a NPN, or a LPN.

### Treating the Tissue the Nerve Innervates

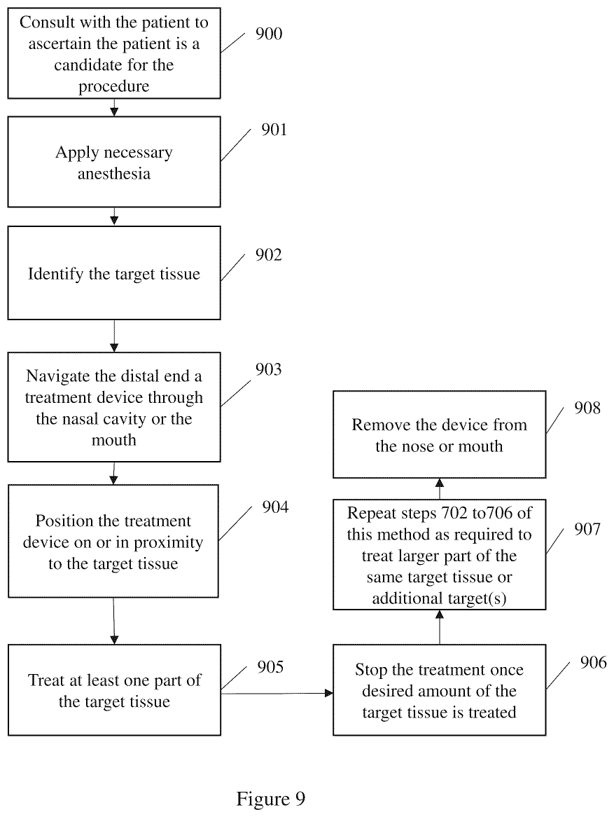

Step by step of one method prescribed for treating diseases is depicted in the flow diagram of FIG. 9:
a. Consult with the patient and ascertain that the patient is a candidate for the current procedure 900,
b. Apply necessary anesthesia to the region of tissue to be treated or put the patient under general anesthesia 901,
c. Identify the target tissue 902,
d. Navigate the distal end a treatment device through the nasal cavity or the mouth 903,
e. Position the treatment device on or in proximity to the desired tissue 904,
f. Treat at least one part of the target tissue 905,
g. Stop the treatment once desired amount of the target tissue is treated 906,
h. Repeat steps 902 to 906 of this method as required to treat larger part of the same target tissue or additional target(s) 907,
i. Remove the device from the nose or mouth 908.

In the method described above, some of the individual steps may be omitted entirely or applied in an alternative order.

In one embodiment of the method, diseases to be treated include symptoms associated sleep disordered breathing, snoring, obstructive sleep apnea (OSA), heart failure and its associated symptoms, hypertension, metabolic diseases such as diabetes, insulin resistance, arrythmia, obesity, nasal and nasopharyngeal symptoms, chronic cough, otologic conditions such as tinnitus, eustachian tube dysfunction, vertigo, Meniere's disease, hearing loss, imbalance, lymphoid tissue hypertrophy, jaw movement disorders such as bruxism, temporomandibular diseases, oral tissue inflammatory diseases such as gingivitis and periodontitis.

In one embodiment target tissue includes at least a portion of nasopharyngeal, oropharyngeal, laryngopharyngeal or palatal mucosa.

In one embodiment the treatment device is a drug delivery device, to deliver pharmaceutical or chemical agents to the target tissue.

In another embodiment the treatment device uses energy such as radiofrequency, microwave, heat, pulsed radiofrequency, cooled radiofrequency, or laser to treat the tissue. In another embodiment the treatment device is a cryogenic device. In one embodiment, the treatment device uses energy to ablate the tissue.

In another embodiment the treatment device is a device that applies pressure on the target tissue. In another embodiment, the treatment device is an expandable balloon.

In one embodiment of the method described herein, the step to ascertain the appropriateness of this therapy includes applying local anesthetics to the target tissue to test the impact of a treat on the disease state.

In one embodiment of the method described herein, the step to identify the target nerve or nerves and the tissue they innervate can include using a nerve tissue visualization aid (e.g., dyes) or other techniques to distinguish nerve tissue from surrounding tissues. In another embodiment, the step to identify target nerve or the tissue it innervates includes methods of nerve stimulation and/or nerve monitoring techniques commonly known in the art. In another method techniques to access the function of a sensory nerve is used to identify the target tissue. In yet another embodiment, methods known in the art is used to visualize the blood vessels and arteries and by association the target nerve and the tissue it innervates.

In one embodiment of the method, the patient is diagnosed with SBD by the physician and a candidate for current procedure. A device used in the procedure is a cryoprobe, capable to reach temperatures between −20° C. to −100° C., using nitrous oxide as cryogen and by employing the Joules Thompson effect. The surgeon uses visualization with or without the help of a navigation instrument to identify the target tissue. To treat, the cryoprobe is navigated through the nasal cavity to reach the nasopharynx. The distal end of the cryoprobe is placed on least a portion of nasopharyngeal mucosa and the ablation mechanism is triggered by allowing the cryogen to flow though the cryoprobe. A portion of the mucosa is then frozen for a period of about 10 seconds to 60 seconds. The flow of cryogen is stopped, and the distal end is allowed to thaw. Once the distal end is released from tissue, the ablation procedure might be repeated on additional locations until desired portion of the mucosa is ablated. Once the ablation is complete, the cryoprobe is navigated out of the nose. In one embodiment of the method, the patient is diagnosed with an otologic condition such as eustachian tube dysfunction. In another embodiment the patient is diagnosed with hypertension. In yet another embodiment, the patient is diagnosed with heart failure. In one embodiment, in addition to nasopharyngeal mucosa, at least a portion of pharyngeal mucosa is also ablated. In another embodiment, the treatment device uses radiofrequency energy to ablate at least a portion of nasopharyngeal mucosa. In other embodiments, the treatment device uses other sources of energy to ablate at least a portion of nasopharyngeal mucosa.

In another embodiment of the method, the patient is diagnosed with SBD by the physician and a candidate for current procedure. To treat the pharyngeal mucosa, including at least a portion of the oro- or laryngopharyngeal mucosa, the cryoprobe is navigated through the oral cavity to reach the pharynx. The distal end of the cryoprobe is placed on least a portion of pharyngeal mucosa and the ablation mechanism is triggered by allowing the cryogen to flow though the cryoprobe. At least a portion of pharyngeal mucosa is then frozen for a period of about 10 seconds to 60 seconds. The flow of cryogen is stopped, and the distal end is allowed to thaw. Once the distal end is released from tissue, the ablation procedure might be repeated on additional locations until desired portion of the mucosa is ablated. Once the ablation is complete, the cryoprobe is navigated out of the oral cavity. In another embodiment the patient is diagnosed with hypertension. In one embodiment, the patient is diagnosed with a metabolic disease such as diabetes or insulin resistance. In yet another embodiment, the patient is diagnosed with heart failure. In one embodiment, the patient is diagnosed with chronic cough. In one embodiment, in addition to pharyngeal mucosa, at least a portion of nasopharyngeal mucosa is also ablated. In another embodiment, the treatment device uses radiofrequency energy to ablate at least a portion of pharyngeal mucosa. In other embodiments, the treatment device uses other sources of energy to ablate at least a portion of pharyngeal mucosa.

In one method of the current application, the patient is diagnosed with SBD by the physician and a candidate for current procedure. A device used in the procedure is an expandable balloon. The surgeon uses visualization with or without the help of a navigation instrument to identify the target tissue. To treat, the balloon is navigated through the nasal cavity to reach the nasopharynx. The distal end of the balloon catheter is placed on least a portion of nasopharyngeal mucosa and the balloon is expanded using an inflation device connected to the proximal end of the treatment device. Once the desired pressure is reached, the balloon is deflated. The procedure might be repeated on additional locations until desired portion of the mucosa is treated. Once the treatment is complete, balloon catheter is navigated out of the nose. In one embodiment of the method, the patient is diagnosed with an otologic condition such as eustachian tube dysfunction. In another embodiment the patient is diagnoses with hypertension. In yet another embodiment, the patient is diagnosed with heart failure. In one embodiment, in addition to nasopharyngeal mucosa, at least a portion of pharyngeal mucosa is also treated.

In one method of the current application, the patient is diagnosed with heart failure by the physician and a candidate for current procedure. A device used in the procedure is an expandable balloon. The surgeon uses visualization with or without the help of a navigation instrument to identify at least a portion of pharyngeal mucosa including at least a portion or oropharyngeal or laryngopharyngeal mucosa. To treat, the balloon is navigated through the oral cavity to reach the pharynx. The distal end of the balloon catheter is placed on least a portion of the pharyngeal mucosa and the balloon is expanded using an inflation device connected to the proximal end of the treatment device. Once the desired pressure is reached, the balloon is deflated. The procedure might be repeated on additional locations until desired portion of the mucosa is treated. Once the treatment is complete, balloon catheter is navigated out of the mouth. In another embodiment the patient is diagnosed with hypertension. In one embodiment, the patient is diagnosed with a metabolic disease such as diabetes or insulin resistance. In yet another embodiment, the patient is diagnosed with heart failure. In one embodiment, the patient is diagnosed with chronic cough. In one embodiment, in addition to pharyngeal mucosa, at least a portion of nasopharyngeal mucosa is also treated.

The applications of the devices and methods discussed above are not limited to the treatments described but may include any number of further treatment applications. Moreover, such devices and methods may be applied to other treatment sites within the body and various treatments may be combined in any number of combinations and order of treatment. Modification of the above-described assemblies and methods for carrying out the invention, combinations between different variations as practicable, and variations of aspects of the invention that are obvious to those of skill in the art are intended to be within the scope of the claims.

What is claimed is:

1. A method for treating dysfunctions or diseases in a patient, comprising:
a. positioning a treatment device having a proximal end, a distal end, an elongate shaft therebetween, and an ablation effector disposed at or near the distal end of the device into proximity of a superior laryngeal nerve or any of its branches; and
b. ablating at least a portion of the superior laryngeal nerve or any of its branches thereof using the ablation effector until at least one symptom of nerve dysfunction or disease is reduced.

2. The method of claim 1 wherein the dysfunctions or diseases to be treated is selected from the group consisting of sleep disorder breathing, sleep apnea, obstructive sleep apnea, snoring, central sleep apnea, heart failure, cardiovascular disease, hypertension, temporomandibular joint disorder (TMD), renal disease, pulmonary disease, diabetes, obesity, dyslipidemia, thyroid disease, such as goiter and hypothyroidism, obesity, neuropsychiatric diseases, metabolic disease, neuroimmune disease, dysphagia, swallowing disorders, gastrointestinal disorders, lower respiratory tract disorders, cough, chronic cough, voice disturbances, otologic conditions such as eustachian tube dysfunction, tinnitus, ear fullness, Meniere's disease, hearing loss, bruxism, gingival and periodontal inflammatory conditions, mouth breathing, anxiety, depression, daytime sleepiness, wheezing, nasopharyngeal symptoms such as postnasal drip, water in the throat, throat dryness, rhinorrhea, nasal obstruction, lymphoid tissue hypertrophy, laryngeal hyperreactivity syndrome, or other disorders that are directly or indirectly associated with hyperreactivity of the aforementioned nerve.

3. The method of claim 1 wherein ablating at least the portion of the superior laryngeal nerve comprises ablating an internal branch of the superior laryngeal nerve.

4. The method of claim 3 wherein positioning the treatment device comprises transcutaneously accessing the internal branch of the superior laryngeal nerve.

5. The method of claim 1 wherein ablating at least the portion of the superior laryngeal nerve comprises ablating at least a portion of oropharyngeal or laryngopharyngeal mucosa.

6. The method of claim 5 wherein ablating at least the portion of oropharyngeal or laryngopharyngeal mucosa comprises applying pressure or mechanical force on the mucosa via the treatment device.

7. The method of claim 6 wherein applying pressure or mechanical force on the mucosa comprises applying the pressure or mechanical force via an expandable balloon catheter.

8. The method of claim 1 wherein ablating at least the portion of the superior laryngeal nerve comprises cryo-ablating via the treatment device.

9. The method of claim 1 wherein ablating at least the portion of the superior laryngeal nerve comprises applying energy to the portion via the treatment device.

10. The method of claim 9 wherein the energy comprises bipolar radiofrequency, pulsed radiofrequency, cooled radiofrequency, microwave, laser, ultrasonic energy, thermal energy, or combinations thereof.

11. The method of claim 1 wherein ablating at least the portion of the superior laryngeal nerve comprises applying a chemical agent to the portion via the treatment device.

12. The method of claim 11 wherein the chemical agent comprises glycerol, ethanol, phenol, or combination thereof.

13. The method of claim 1 wherein ablating at least the portion of the superior laryngeal nerve comprises applying a therapeutic agent to the portion via the treatment device.

14. The method of claim 13 wherein therapeutics agent comprises TRP channel inhibitors, TRPA1 and TRPV1 antagonist, substance P antagonist, CGRP antagonist, P2×3 antagonist, P2×2 antagonists, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin, neurotoxin agents, anesthetics, analgesics, corticosteroids, VIP antagonist, muscarinic antagonists, nicotinic antagonists, anticholinergic agents, or combinations thereof, beta blockers, beta-2 (β2) agonists and alpha-2 (α2) agonists.

15. The method of claim 1 further comprising visualizing at least the portion of the superior laryngeal nerve while ablating.

16. The method of claim 1 further comprising illuminating at least the portion of the superior laryngeal nerve while ablating.

17. A method for treating dysfunctions or diseases in a patient, comprising:
a. introducing a treatment device into a nasopharynx of the patient, the treatment device having a proximal end, a distal end, an elongate shaft therebetween, and an ablation effector disposed at or near the distal end of the device;
b. advancing the distal end of the device under visualization into proximity of a palatovaginal canal (PVC) opening within the nasopharynx; and
c. ablating all or substantially all of the pharyngeal nerve fibers passing through the PVC opening using the ablation effector until at least one symptom of nerve dysfunction or disease is reduced.

18. The method of claim 17 wherein the dysfunctions or diseases to be treated is selected from the group consisting of sleep disorder breathing, sleep apnea, obstructive sleep apnea, snoring, central sleep apnea, heart failure, cardiovascular disease, hypertension, temporomandibular joint disorder (TMD), renal disease, pulmonary disease, diabetes, obesity, dyslipidemia, thyroid disease, such as goiter and hypothyroidism, obesity, neuropsychiatric diseases, metabolic disease, neuroimmune disease, dysphagia, swallowing disorders, gastrointestinal disorders, lower respiratory tract disorders, cough, chronic cough, voice disturbances, otologic conditions such as eustachian tube dysfunction, tinnitus, ear fullness, Meniere's disease, hearing loss, otitis media, chronic otitis media, otitis media with effusion, chronic suppurative otitis media, bruxism, gingival and periodontal inflammatory conditions, mouth breathing, anxiety, depression, daytime sleepiness, wheezing, nasopharyngeal symptoms such as postnasal drip, water in the throat, throat dryness, rhinorrhea, nasal obstruction, lymphoid tissue hypertrophy, laryngeal hyperreactivity syndrome, or other disorders that are directly or indirectly associated with hyperreactivity of the aforementioned nerve.

19. The method of claim 17 wherein ablating all or substantially all of the pharyngeal nerve fibers comprises applying pressure or mechanical force on a mucosa covering the PVC opening within the nasopharynx.

20. The method of claim 19 wherein applying pressure or mechanical force comprises applying the pressure or mechanical force via an expandable balloon catheter.

21. The method of claim 17 wherein ablating all or substantially all of the pharyngeal nerve fibers comprises cryo-ablating via the treatment device.

22. The method of claim 17 wherein ablating all or substantially all of the pharyngeal nerve fibers comprises applying energy to the pharyngeal nerve fibers via the treatment device.

23. The method of claim 22 wherein the energy comprises bipolar radiofrequency, pulsed radiofrequency, cooled radiofrequency, microwave, laser, ultrasonic energy, thermal energy, or combinations thereof.

24. The method of claim 17 wherein ablating all or substantially all of the pharyngeal nerve fibers comprises applying a chemical agent to the pharyngeal nerve fibers.

25. The method of claim 24 wherein the chemical agent comprises glycerol, ethanol, phenol, or combination thereof.

26. The method of claim 17 wherein ablating all or substantially all of the pharyngeal nerve fibers comprises applying a therapeutic agent to the pharyngeal nerve fibers.

27. The method of claim 26 wherein therapeutics agent comprises TRP channel inhibitors, TRPA1 and TRPV1 antagonist, substance P antagonist, CGRP antagonist, P2×3 antagonist, P2×2 antagonists, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin, neurotoxin agents, anesthetics, analgesics, corticosteroids, VIP antagonist, muscarinic antagonists, nicotinic antagonists, anticholinergic agents, or combinations thereof, beta blockers, beta-2 (β2) agonists and alpha-2 (α2) agonists.

28. The method of claim 17 further comprising visualizing the pharyngeal nerve fibers while ablating.

29. The method of claim 17 further comprising illuminating the pharyngeal nerve fibers while ablating.

30. A method for treating dysfunctions or diseases in a patient, comprising:
a. introducing a treatment device into the oral or nasal cavity of the patient, the treatment device having a proximal end, a distal end, an elongate shaft therebetween, and an ablation effector disposed at or near the distal end of the device;
b. ablating all or substantially all of the lesser palatine nerve fibers using the ablation effector until at least one symptom of nerve dysfunction or disease is reduced.

31. The method of claim 30 wherein the dysfunctions or diseases to be treated is selected from the group consisting of sleep disorder breathing, sleep apnea, obstructive sleep apnea, snoring, central sleep apnea, heart failure, cardiovascular disease, hypertension, temporomandibular joint disorder (TMD), renal disease, pulmonary disease, diabetes, obesity, dyslipidemia, thyroid disease, such as goiter and hypothyroidism, obesity, neuropsychiatric diseases, metabolic disease, neuroimmune disease, dysphagia, swallowing disorders, gastrointestinal disorders, lower respiratory tract disorders, cough, chronic cough, voice disturbances, otologic conditions such as eustachian tube dysfunction, tinnitus, ear fullness, Meniere's disease, hearing loss, bruxism, gingival and periodontal inflammatory conditions, mouth breathing, anxiety, depression, daytime sleepiness, wheezing, nasopharyngeal symptoms such as postnasal drip, water in the throat, throat dryness, rhinorrhea, nasal obstruction, lymphoid tissue hypertrophy, laryngeal hyperreactivity syndrome, or other disorders that are directly or indirectly associated with hyperreactivity of the aforementioned nerve.

32. The method of claim 30 wherein introducing the treatment device comprises introducing through the nasal cavity.

33. The method of claim 32 wherein introducing the treatment device through the nasal cavity comprises advancing the distal end of the treatment device into the maxillary sinus of the patient to the proximity of lesser palatine canal.

34. The method of claim 30 wherein introducing the treatment device comprises introducing through the oral cavity.

35. The method of claim 34 wherein introducing the treatment device through the oral cavity comprises advancing the distal end of the treatment device into the proximity of at least one lesser palatine foramen.

36. The method of claim 30 wherein ablating all or substantially all of the lesser palatine fibers comprises ablating at least a portion of a soft palate mucosa.

37. The method of claim 30 wherein ablating all or substantially all of the lesser palatine fibers comprises ablating at least a portion of levator veli palatini, palatopharyngeus, or musculus uvula.

38. The method of claim 30 wherein ablating all or substantially all of the lesser palatine fibers comprises cryo-ablating via the treatment device.

39. The method of claim 30 wherein ablating all or substantially all of the lesser palatine fibers comprises applying energy to the lesser palatine fibers.

40. The method of claim 39 wherein the energy comprises bipolar radiofrequency, pulsed radiofrequency, cooled radiofrequency, microwave, laser, ultrasonic energy, thermal energy, or combinations thereof.

41. The method of claim 30 wherein ablating all or substantially all of the lesser palatine fibers comprises applying a chemical agent to the lesser palatine fibers.

42. The method of claim 41 wherein the chemical agent comprises glycerol, ethanol, phenol, or combination thereof.

43. The method of claim 30 wherein ablating all or substantially all of the lesser palatine fibers comprises applying a therapeutic agent to the lesser palatine fibers.

44. The method of claim 43 wherein therapeutics agent comprises TRP channel inhibitors, TRPA1 and TRPV1 antagonist, substance P antagonist, CGRP antagonist, P2×3 antagonist, P2×2 antagonists, voltage-gated sodium channel inhibitors, Piezo receptor antagonists, 5HT receptor antagonist, neurokinin receptor antagonists, sodium channel inhibitors, nerve growth factor receptor antagonist, capsaicin or its analogues, botulinum toxin, neurotoxin agents, anesthetics, analgesics, corticosteroids, VIP antagonist, muscarinic antagonists, nicotinic antagonists, anticholinergic agents, or combinations thereof. beta blockers, beta-2 (β2) agonists and alpha-2 (α2) agonists.

45. The method of claim 30 further comprising visualizing the lesser palatine fibers while ablating.
